(19) United States
(12) Patent Application Publication
(10) Pub. No.: US 2023/0071245 A1
(43) Pub. Date: Mar. 9, 2023

(54) COMPOSITIONS AND METHODS FOR CONTROLLING UNDESIRABLE MICROBES AND IMPROVING ANIMAL HEALTH

(71) Applicant: AgBiome, Inc., Durham, NC (US)

(72) Inventors: Matthew Bryon Biggs, Durham, NC (US); James Michael Kremer, Durham, NC (US); Vadim Beilinson, Cary, NC (US); Dharanesh Mahimapura Gangaiah, Fishers, IN (US); Kelly Craig, Durham, NC (US)

(73) Assignee: AgBiome, Inc., Durham, NC (US)

(21) Appl. No.: 17/793,249

(22) PCT Filed: Jan. 15, 2021
(86) PCT No.: PCT/US2021/013675

§ 371 Date: Jul. 15, 2022

Related U.S. Application Data
(60) Provisional application No. 63/094,023, filed on Oct. 20, 2020.
(60) Provisional application No. 63/053,142, filed on Jul. 17, 2020.
(60) Provisional application No. 63/047,569, filed on Jul. 2, 2020.
(60) Provisional application No. 63/040,286, filed on Jun. 17, 2020.
(60) Provisional application No. 62/970,907, filed on Feb. 6, 2020.
(60) Provisional application No. 62/962,419, filed on Jan. 17, 2020.

(51) Int. Cl. A61K 35/742; A61K 35/745; A61K 35/747; A61K 35/744; A61P 31/04; A61K 35/741

(52) U.S. Cl. CPC A61K 35/742; A61K 35/745; A61K 35/747; A61K 35/744; A61P 31/04; A61K 35/741

(57) ABSTRACT

Compositions and methods for controlling undesirable microbes, such as pathogenic bacteria, are provided. Such compositions and methods comprise a bacterial strain that can be used as a probiotic for animals. Therefore, methods for controlling undesirable microbes, such as pathogenic bacteria, in the gastrointestinal tract, for example, of an animal and methods for improving animal health are also provided.

### FIELD OF THE INVENTION

The invention relates to the use of bacterial strains to inhibit the growth of pathogenic bacteria and to promote animal health.

### BACKGROUND

An animal's gastrointestinal tract is constantly challenged by bacteria, viruses, and protozoa found in feed, bedding, and the environment. The gastrointestinal tract counters these undesirable microbes using physical, chemical, and immunological defenses. These defenses also include beneficial microbes, such as bacteria, that are resident in the gastrointestinal tract. Pathogens, stress, metabolic upset, the use of antimicrobials, and other causes may upset the balance of the intestinal microbiome, which can affect digestion and can also make the animal more susceptible to disease. Providing the animal with beneficial bacteria that assist in the establishment or reestablishment of a healthy intestinal microbiome profile can help maintain healthy animals and maximize animal performance.

### SUMMARY

Compositions and methods for controlling undesirable microbes are provided. Such compositions and methods comprise beneficial bacterial strains that control undesirable microbes, including pathogenic bacterial strains. Such pathogenic bacterial strains include one or more strains of Salmonella spp. and/or Escherichia coli. The beneficial bacterial strains can be used as probiotics or direct-fed microbials (DFMs) for animals. The beneficial bacterial strains may also be used in the absence of disease to promote animal health and to establish or maintain a healthy intestinal microbiome profile. Also provided herein are methods for formulating and administering the beneficial bacterial strains to animals for improving animal health and performance and/or for treating or preventing disease caused by undesirable microbes in the gut.

### BRIEF DESCRIPTION OF THE DRAWINGS

FIG. 1 provides graphs showing average daily gain (ADG), average daily feed intake (ADFI), and feed efficiency (determined by the ratio of gain to feed (G:F)), as a measure of overall growth performance from proof of principle (POP) animal study POP1. ** indicates p<0.001; * indicates p<0.050; ˜ indicates p<0.1

FIG. 2 provides graphs showing average daily gain (ADG), average daily feed intake (ADFI), and feed efficiency (determined by the ratio of gain to feed (G:F)), as a measure of overall growth performance from proof of principle (POP) animal study POP2. * indicates p<0.1; ** indicates p<0.01; *** indicates p <0.001

FIG. 3 provides graphs showing average daily gain (ADG), average daily feed intake (ADFI), and feed efficiency (determined by the ratio of gain to feed (G:F)), as a measure of overall growth performance from proof of principle (POP) animal study POP3.

### DETAILED DESCRIPTION

### I. Overview

Compositions and methods for controlling undesirable microbes are provided. The compositions and methods provided herein involve the use of beneficial bacterial strains that can control undesirable microbes, such as pathogenic bacteria, and can promote animal health.

Bacterial strains of the invention and variants thereof retain the ability to control one or more undesirable microbes and/or improve digestion of certain types of feed. In some embodiments, bacterial strains of the invention and variants thereof will retain the ability to control harmful, disease-causing, or pathogenic bacteria. Pathogenic bacteria includes pathogenic strains of Clostridia spp, (such as C. perfringens and C. dificile) Salmonella spp (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli), Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp. and the like.

The term “controlling” undesirable microbes refers to one or more of inhibiting or reducing the growth, feeding, reproduction, and/or proliferation of an undesirable microbe or its population, or killing (e.g., causing the morbidity or mortality, or reduced/stopped proliferation) of an undesirable microbe or its population. Without being bound by theory, the beneficial bacteria may be able to control the undesirable microbe by secretion of a toxic metabolite, or by out-competing and/or out-growing the undesirable microbe (“competitive exclusion”). Control may be by promotion of the growth of beneficial bacteria. In some embodiments, strains of the invention and variants thereof will retain the ability to control pathogenic bacteria. In further embodiments, strains of the invention and variants thereof will retain the ability to control Salmonella spp, and/or Escherichia spp. In some embodiments, pathogenic bacteria may be reduced by about 0.5 log, about 1 log, about 2 log, about 3 log, about 4 log, about 5 log, or more.

The beneficial bacterial strains of the invention may be utilized in products, such as probiotics, which aim to control undesirable microbes, including pathogenic bacteria. The term “probiotics” has been defined by the Food and Agriculture Organization of the United Nations (FAO) and World Health Organization (WHO) as live microorganisms which when administered in adequate amounts confer a health benefit on the host. Probiotics include beneficial bacteria. Direct-fed microbial (DFM) products are probiotic products that contain live, viable microorganisms, particularly beneficial bacteria, which deliver these bacteria to the gastrointestinal tract for colonization of the tract to improve the health and performance of the animal. DFMs influence the gut microbiome in a positive way by supporting the growth of beneficial microbes, such as beneficial bacteria, and/or by controlling undesirable microbes, such as pathogenic bacteria. Additionally, beneficial bacteria may promote animal health by acidifying the gut, thereby creating an environment that disfavors colonization by undesirable microbes, and/or may aid in the digestion of specific feed ingredients, for example dried distillers grains with solubles (DDGS), which is a high-protein livestock feed. Beneficial bacteria are also known to stimulate the immune system of the host animal. Further, beneficial bacteria may produce antimicrobial substances which can control undesirable microbes. Such antimicrobial substances include bacteriocins, lipopeptides such as iturins and surfactin, and short chain fatty acids (SCFAs) such as acetic and lactic acid.

### II. Bacterial Strains

Various bacterial strains are provided which can be used singly or in combination to control one or more undesirable microbes and/or improve animal health and performance. Such bacterial strains include the Bacillus spp. strains AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof. Cell populations comprising one or more of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374 are provided, as well as populations of spores derived from each of these strains, or any preparation thereof. Thus, various bacterial strains and/or feed compositions provided herein comprise as an active ingredient a cell population, spore, forespore, or combination thereof, or a supernatant, fermentation product, filtrate or extract thereof of one or more of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof.

AIP088262 was deposited with the International Depository Authority of the Agricultural Research Culture Collection (NRRL), 1815 North University Street, Peoria, Ill. 61604 U.S.A. on Jan. 17, 2020 and assigned NRRL No. B-67923.

AIP068104 was deposited with the International Depository Authority of the Agricultural Research Culture Collection (NRRL), 1815 North University Street, Peoria, Ill. 61604 U.S.A. on Jan. 17, 2020 and assigned NRRL No. B-67922.

AIP016597 was deposited with the International Depository Authority of the Agricultural Research Culture Collection (NRRL), 1815 North University Street, Peoria, Ill. 61604 U.S.A. on Jan. 17, 2020 and assigned NRRL No. B-67921.

AIP004816 was deposited with the International Depository Authority of the Agricultural Research Culture Collection (NRRL), 1815 North University Street, Peoria, Ill. 61604 U.S.A. on Jan. 17, 2020 and assigned NRRL No. B-67920.

AIP053802 was deposited with the International Depository Authority of the Agricultural Research Culture Collection (NRRL), 1815 North University Street, Peoria, Ill. 61604 U.S.A. on Jan. 17, 2020 and assigned NRRL No. B-67919.

AIP004634 was deposited with the International Depository Authority of the Agricultural Research Culture Collection (NRRL), 1815 North University Street, Peoria, Ill. 61604 U.S.A. on Jan. 17, 2020 and assigned NRRL No. B-67918.

AIP006035 was deposited with the International Depository Authority of the Agricultural Research Culture Collection (NRRL), 1815 North University Street, Peoria, Ill. 61604 U.S.A. on Jan. 17, 2020 and assigned NRRL No. B-67917.

AIP029002 was deposited with the International Depository Authority of the Agricultural Research Culture Collection (NRRL), 1815 North University Street, Peoria, Ill. 61604 U.S.A. on Jan. 17, 2020 and assigned NRRL No. B-67916.

AIP066414 was deposited with the International Depository Authority of the Agricultural Research Culture Collection (NRRL), 1815 North University Street, Peoria, Ill. 61604 U.S.A. on Jan. 17, 2020 and assigned NRRL No. B-67915.

AIP093093 was deposited with the International Depository Authority of the Agricultural Research Culture Collection (NRRL), 1815 North University Street, Peoria, Ill. 61604 U.S.A. on Jan. 17, 2020 and assigned NRRL No. B-67914.

AIP022568 was deposited with the International Depository Authority of the Agricultural Research Culture Collection (NRRL), 1815 North University Street, Peoria, Ill. 61604 U.S.A. on Jan. 17, 2020 and assigned NRRL No. B-67913.

AIP032005 was deposited with the International Depository Authority of the Agricultural Research Culture Collection (NRRL), 1815 North University Street, Peoria, Ill. 61604 U.S.A. on Jan. 17, 2020 and assigned NRRL No. B-67912.

AIP012656 was deposited with the International Depository Authority of the Agricultural Research Culture Collection (NRRL), 1815 North University Street, Peoria, Ill. 61604 U.S.A. on Jun. 22, 2020 and assigned NRRL No. B-67968.

AIP002364 was deposited with the International Depository Authority of the Agricultural Research Culture Collection (NRRL), 1815 North University Street, Peoria, Ill. 61604 U.S.A. on Jun. 22, 2020 and assigned NRRL No. B-67967.

AIP044543 was deposited with the International Depository Authority of the Agricultural Research Culture Collection (NRRL), 1815 North University Street, Peoria, Ill. 61604 U.S.A. on Jun. 22, 2020 and assigned NRRL No. B-67965.

AIP090377 was deposited with the International Depository Authority of the Agricultural Research Culture Collection (NRRL), 1815 North University Street, Peoria, Ill. 61604 U.S.A. on Jun. 22, 2020 and assigned NRRL No. B-67964.

AIP048352 was deposited with the International Depository Authority of the Agricultural Research Culture Collection (NRRL), 1815 North University Street, Peoria, Ill. 61604 U.S.A. on Jun. 22, 2020 and assigned NRRL No. B-67966.

AIP089343 was deposited with the International Depository Authority of the Agricultural Research Culture Collection (NRRL), 1815 North University Street, Peoria, Ill. 61604 U.S.A. on Jun. 22, 2020 and assigned NRRL No. B-67962.

AIP007305 was deposited with the International Depository Authority of the Agricultural Research Culture Collection (NRRL), 1815 North University Street, Peoria, Ill. 61604 U.S.A. on Jun. 22, 2020 and assigned NRRL No. B-67961.

AIP033189 was deposited with the International Depository Authority of the Agricultural Research Culture Collection (NRRL), 1815 North University Street, Peoria, Ill. 61604 U.S.A. on Jun. 22, 2020 and assigned NRRL No. B-67958.

AIP063641 was deposited with the International Depository Authority of the Agricultural Research Culture Collection (NRRL), 1815 North University Street, Peoria, Ill. 61604 U.S.A. on Jun. 22, 2020 and assigned NRRL No. B-67963.

AIP087760 was deposited with the International Depository Authority of the Agricultural Research Culture Collection (NRRL), 1815 North University Street, Peoria, Ill. 61604 U.S.A. on Jun. 22, 2020 and assigned NRRL No. B-67970.

AIP097873 was deposited with the International Depository Authority of the Agricultural Research Culture Collection (NRRL), 1815 North University Street, Peoria, Ill. 61604 U.S.A. on Jun. 22, 2020 and assigned NRRL No. B-67960.

AIP056374 was deposited with the International Depository Authority of the Agricultural Research Culture Collection (NRRL), 1815 North University Street, Peoria, Ill. 61604 U.S.A. on Jun. 22, 2020 and assigned NRRL No. B-67959.

Each of the deposits identified above will be maintained under the terms of the Budapest Treaty on the International Recognition of the Deposit of Microorganisms for the Purposes of Patent Procedure. Each deposit was made merely as a convenience for those of skill in the art and is not an admission that a deposit is required under 35 U.S.C. § 112.

The term “isolated” encompasses a bacterium, spore, or other entity or substance, that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or more of the other components with which they were initially associated.

As used herein, a substance is “pure” if it is substantially free of other components. The terms “purify,” “purifying” and “purified” refer to a bacterium, spore, or other material that has been separated from at least some of the components with which it was associated either when initially produced or generated (e.g., whether in nature or in an experimental setting), or during any time after its initial production. A bacterium or spore or a bacterial population or a spore population may be considered purified if it is isolated at or after production, such as from a material or environment containing the bacterium or bacterial population or spore, and a purified bacterium or bacterial population or spore may contain other materials up to about 10%, about 20%, about 30%, about 40%, about 50%, about 60%, about 70%, about 80%, about 90%, or above about 90% and still be considered purified. In some embodiments, purified bacteria or spores and bacterial populations or spore populations are more than about 80%, about 85%, about 90%, about 91%, about 92%, about 93%, about 94%, about 95%, about 96%, about 97%, about 98%, about 99%, or more than about 99% pure. In specific embodiments, a culture of bacteria contains no other bacterial species in quantities to be detected by normal bacteriological techniques.

In some embodiments, the compositions of the invention comprise substantially pure cultures of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374. The compositions of the invention also provide progeny of substantially pure cultures of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, wherein the culture has all of the physiological and morphological characteristics of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, respectively. By “population” is intended a group or collection that comprises two or more individuals (i.e., 10, 100, 1,000, 10,000, 1×10⁶, 1×10⁷, or 1×10⁸ or greater) of a given bacterial strain. Various compositions are provided herein that comprise a population of at least one bacterial strain or a mixed population of individuals from more than one bacterial strain. In specific embodiments, the population of at least one of a bacterial strain (i.e., cells of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374, or an active variant of any thereof, or spores or forespores or a combination of cells, forespores and/or spores, formed from one or more of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374, or an active variant of any thereof) comprises a concentration of at least about 10³ CFU/ml to about 10¹⁵ CFU/ml, about 10³CFU/ml to about 10¹² CFU/ml, about 10³ CFU/ml to about 10¹⁰ CFU/ml, about 10⁴ CFU/ml to about 10⁸ CFU/ml, about 10³ CFU/ml to about 10⁴ CFU/ml, about 10⁴ CFU/ml to about 10⁵ CFU/ml, about 10⁵ CFU/ml to about 10⁶ CFU/ml, about 10⁶ CFU/ml to about 10⁷ CFU/ml, about 10⁷ CFU/ml to about 10⁸ CFU/ml, about 10⁸ CFU/ml to about 10⁹ CFU/ml, about 10⁹ CFU/ml to about 10¹⁰ CFU/ml, about 10¹⁰ CFU/ml to about 10¹¹ CFU/ml, about 10¹¹ CFU/ml to about 10¹² CFU/ml, about 10¹² CFU/ml to about 10¹³ CFU/ml, about 10¹³ CFU/ml to about 10¹⁴ CFU/ml, or about 10¹⁴ CFU/ml to about 10¹⁵ CFU/ml. In other embodiments, the concentration of the bacterial strain or combination thereof provided herein or active variant thereof comprises or consists of at least about 10³ CFU/ml, at least about 10⁴ CFU/ml, at least about 10⁵CFU/ml, at least about 10⁶ CFU/ml, at least about 10⁷ CFU/ml, at least about 10⁸ CFU/ml, at least about 10⁹ CFU/ml, at least about 10¹⁰ CFU/ml, at least about 10¹¹ CFU/ml, at least about 10¹² CFU/ml, at least about 10¹³ CFU/ml, at least about 10¹⁴ CFU/ml, or at least about 10¹⁵ CFU/ml.

A “spore” refers to at least one dormant (at application) but viable reproductive unit of a bacterial species. Non-limiting methods by which spores are formed from each of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374 (or variants of any thereof) are disclosed elsewhere herein. It is further recognized the populations disclosed herein can comprise a combination of vegetative cells and forespores (cells in an intermediate stage of spore formation); a combination of forespores and spores; or a combination of forespores, vegetative cells and/or spores.

As used herein, “derived from” means directly isolated or obtained from a particular source or alternatively having identifying characteristics of a substance or organism isolated or obtained from a particular source. In the event that the “source” is an organism, “derived from” means that it may be isolated or obtained from the organism itself, as a fermentation product, or from the culture broth, suspension, or medium used to culture or grow said organism. A compound or composition “derived from” or “obtainable from” means that the compound or composition may be isolated from or produced by a cell culture or a whole cell broth, or suspension, filtrate, supernatant, fraction, or extract derived from a cell culture or a whole cell broth.

As used herein, “whole broth culture” or “whole cell broth” refers to a liquid culture containing both cells and media. If bacteria are grown on a plate, the cells can be harvested in water or other liquid, whole culture. The terms “whole broth culture” and “whole cell broth” are used interchangeably.

As used herein, “supernatant” refers to the liquid remaining when cells grown in broth or are harvested in another liquid from an agar plate and are removed by centrifugation, filtration, sedimentation, or other means well known in the art. In some embodiments, the supernatant may be diluted with another composition, such as water, buffer, fresh media, and/or a formulation. The diluted supernatant is still considered a supernatant of the invention.

As used herein, “filtrate” refers to liquid from a whole broth culture that has passed through a membrane. The filtrate may comprise a concentrated amount of an effective compound or metabolite compared to the concentration of the effective compound or metabolite in the whole broth culture or supernatant. As used herein, “extract” refers to liquid substance removed from cells by a solvent (water, detergent, buffer, and/or organic solvent, for example) and separated from the cells by centrifugation, filtration, or other method known in the art. The extract may comprise a concentrated amount of an effective compound or metabolite compared to the concentration of the effective compound or metabolite in the cells prior to extraction. Alternatively, the filtrate or extract may then be diluted with another composition, such as water, buffer, fresh media, and/or a formulation. Such diluted filtrates or extracts are still considered filtrates and extracts of the invention.

As used herein, “fermentation product” refers to a compound, substance, or byproduct of fermentation of a bacterial strain (i.e., at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof). A fermentation product may be soluble or insoluble. An effective compound or metabolite is a fermentation product or compound present in the supernatant, whole cell broth, or bacterial strain which may control one or more undesirable microbes and/or aid in the digestion of some types of feed.

In some embodiments, a composition of the invention comprises a fermentation product, filtrate, or extract derived from a bacterial strain or a combination of bacterial strains cultured together, wherein the composition comprises a concentrated amount of an effective compound or metabolite compared to the amount in a whole cell broth or supernatant of the bacterial strain, wherein the bacterial strain is at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof. In other embodiments, a composition of the invention comprises a diluted fermentation product, diluted filtrate, diluted extract, or diluted supernatant derived from a bacterial strain, wherein the composition comprises a diluted amount of the effective compound or metabolite compared to the amount in a whole cell broth or undiluted supernatant of the bacterial strain or combination of bacterial strains cultured together, wherein the bacterial strain is at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof The diluted fermentation product, diluted filtrate, diluted extract, or diluted supernatant may still comprise an effective amount of the effective compound or metabolite.

The compositions and methods described herein comprise or are derived from a bacterial strain or combination of bacterial strains (i.e., at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a spore or a forespore or a combination of cells, forespores or/and spores, from any one of AIP088262, AIP068104,

AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof). Methods comprise cultivating at least one of these bacterial strains. In some embodiments, at least one of these bacterial strains is cultivated and compounds and/or compositions are obtained by isolating these compounds and/or compositions from the culture of at least one of these bacterial strains.

The compositions described herein may comprise or may be derived from a combination of bacterial strains selected from AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a cell, a spore, or a forespore or a combination of cells, forespores or/and spores, from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof The combination of bacterial strains may comprise at least two bacterial strains of the invention, at least three, at least four, at least five, at least 6, at least 7, at least 8, at least 9, or at least 10 bacterial strains of the invention. In some embodiments, the combination of at least three, at least four, at least five, at least 6, at least 7, at least 8, at least 9, or at least 10 bacterial strains comprises at least a combination of AIP004816 and AIP053802. In some embodiments, the combination of at least three, at least four, at least five, at least 6, at least 7, at least 8, at least 9, or at least 10 bacterial strains comprises at least a combination of AIP004816 and AIP097873. In some embodiments, the combination of at least three, at least four, at least five, at least 6, at least 7, at least 8, at least 9, or at least 10 bacterial strains comprises at least a combination of AIP004816 and AIP012656. In some embodiments, the combination of at least three, at least four, at least five, at least 6, at least 7, at least 8, at least 9, or at least 10 bacterial strains comprises at least a combination of AIP053802 and AIP087760. In some embodiments, the combination of at least three, at least four, at least five, at least 6, at least 7, at least 8, at least 9, or at least 10 bacterial strains comprises at least a combination of AIP006035 and AIP097873. In some embodiments, the combination of at least three, at least four, at least five, at least 6, at least 7, at least 8, at least 9, or at least 10 bacterial strains comprises at least a combination of AIP006035 and AIP053802. In some embodiments, the combination of at least four, at least five, at least 6, at least 7, at least 8, at least 9, or at least 10 bacterial strains comprises at least a combination of AIP006035, AIP004816, and AIP053802.

In specific embodiments the composition comprises a combination of strains AIP088262 and AIP022568. In specific embodiments the composition comprises a combination of strains AIP088262 and AIP004816. In specific embodiments the composition comprises a combination of strains AIP088262 and AIP053802. In specific embodiments the composition comprises a combination of strains AIP088262 and AIP004634. In specific embodiments the composition comprises a combination of strains AIP088262 and AIP006035. In specific embodiments the composition comprises a combination of strains AIP088262 and AIP012656. In specific embodiments the composition comprises a combination of strains AIP088262 and AIP044543. In specific embodiments the composition comprises a combination of strains AIP088262 and AIP033189. In specific embodiments the composition comprises a combination of strains AIP088262 and AIP087760. In specific embodiments the composition comprises a combination of strains AIP088262 and AIP097873. In specific embodiments the composition comprises a combination of strains AIP022568 and AIP004816. In specific embodiments the composition comprises a combination of strains AIP022568 and AIP053802. In specific embodiments the composition comprises a combination of strains AIP022568 and AIP004634. In specific embodiments the composition comprises a combination of strains AIP022568 and AIP006035. In specific embodiments the composition comprises a combination of strains AIP022568 and AIP012656. In specific embodiments the composition comprises a combination of strains AIP022568 and AIP044543. In specific embodiments the composition comprises a combination of strains AIP022568 and AIP033189. In specific embodiments the composition comprises a combination of strains AIP022568 and AIP087760. In specific embodiments the composition comprises a combination of strains AIP022568 and AIP097873. In specific embodiments the composition comprises a combination of strains AIP004816 and AIP053802. In specific embodiments the composition comprises a combination of strains AIP004816 and AIP004634. In specific embodiments the composition comprises a combination of strains AIP004816 and AIP006035. In specific embodiments the composition comprises a combination of strains AIP004816 and AIP012656. In specific embodiments the composition comprises a combination of strains AIP004816 and AIP044543. In specific embodiments the composition comprises a combination of strains AIP004816 and AIP033189. In specific embodiments the composition comprises a combination of strains AIP004816 and AIP087760. In specific embodiments the composition comprises a combination of strains AIP004816 and AIP097873. In specific embodiments the composition comprises a combination of strains AIP053802 and AIP004634. In specific embodiments the composition comprises a combination of strains AIP053802 and AIP006035. In specific embodiments the composition comprises a combination of strains AIP053802 and AIP012656. In specific embodiments the composition comprises a combination of strains AIP053802 and AIP044543. In specific embodiments the composition comprises a combination of strains AIP053802 and AIP033189. In specific embodiments the composition comprises a combination of strains AIP053802 and AIP087760. In specific embodiments the composition comprises a combination of strains AIP053802 and AIP097873. In specific embodiments the composition comprises a combination of strains AIP004634 and AIP006035. In specific embodiments the composition comprises a combination of strains AIP004634 and AIP012656. In specific embodiments the composition comprises a combination of strains AIP004634 and AIP044543. In specific embodiments the composition comprises a combination of strains AIP004634 and AIP033189. In specific embodiments the composition comprises a combination of strains AIP004634 and AIP087760. In specific embodiments the composition comprises a combination of strains AIP004634 and AIP097873. In specific embodiments the composition comprises a combination of strains AIP006035 and AIP012656. In specific embodiments the composition comprises a combination of strains AIP006035 and AIP044543. In specific embodiments the composition comprises a combination of strains AIP006035 and AIP033189. In specific embodiments the composition comprises a combination of strains AIP006035 and AIP087760. In specific embodiments the composition comprises a combination of strains AIP006035 and AIP097873. In specific embodiments the composition comprises a combination of strains AIP012656 and AIP044543. In specific embodiments the composition comprises a combination of strains AIP012656 and AIP033189. In specific embodiments the composition comprises a combination of strains AIP012656 and AIP087760. In specific embodiments the composition comprises a combination of strains AIP012656 and AIP097873. In specific embodiments the composition comprises a combination of strains AIP044543 and AIP033189. In specific embodiments the composition comprises a combination of strains AIP044543 and AIP087760. In specific embodiments the composition comprises a combination of strains AIP044543 and AIP097873. In specific embodiments the composition comprises a combination of strains AIP033189 and AIP087760. In specific embodiments the composition comprises a combination of strains AIP033189 and AIP097873. In specific embodiments the composition comprises a combination of strains AIP087760 and AIP097873.

Moreover, in specific embodiments the composition comprises a combination of strains AIP088262 and one or more of any of AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP068104 and one or more of any of AIP088262, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP016597 and one or more of any of AIP088262, AIP068104, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP004816 and one or more of any of AIP088262, AIP068104, AIP016597, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP053802 and one or more of any of AIP088262, AIP068104, AIP016597, AIP004816, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP004634 and one or more of any AIP088262, AIP068104, AIP016597, AIP004816, AIP053802, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP006035 and one or more of any of AIP088262, AIP068104, AIP016597, AIP004816, AIP053802, AIP004634, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP029002 and one or more of any of AIP088262, AIP068104, AIP016597, AIP004816, AIP053802, AIP004634, AIP006035, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP066414 and one or more of any of AIP088262, AIP068104, AIP016597, AIP004816, AIP053802, AIP004634, AIP006035, AIP029002, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP093093 and one or more of any of AIP088262, AIP068104, AIP016597, AIP004816, AIP053802, AIP004634, AIP006035, AIP029002, AIP066414, AIP022568, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP022568 and one or more of any of AIP088262, AIP068104, AIP016597, AIP004816, AIP053802, AIP004634, AIP006035, AIP029002, AIP066414, AIP093093, AIP032005, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP032005 and one or more of any of AIP088262, AIP068104, AIP016597, AIP004816, AIP053802, AIP004634, AIP006035, AIP029002, AIP066414, AIP093093, AIP022568, AIP012656, AIP002364, AIP044543, AIP090377, AIP048352, AIP089343, AIP007305, AIP033189, AIP063641, AIP087760, AIP097873, AIP056374. In specific embodiments the composition comprises a combination of strains AIP012656 and one or more of any of AIP088262, AIP068104, AIP016597, AIP004816, AIP053802, AIP004634, AIP006035, AIP029002, AIP066414, AIP093093, AIP022568, AIP032005, AIP002364, AIP044543, AIP090377, AIP048352, AIP089343, AIP007305, AIP033189, AIP063641, AIP087760, AIP097873, AIP056374. In specific embodiments the composition comprises a combination of strains AIP002364 and one or more of any of AIP088262, AIP068104, AIP016597, AIP004816, AIP053802, AIP004634, AIP006035, AIP029002, AIP066414, AIP093093, AIP022568, AIP032005, AIP012656, AIP044543, AIP090377, AIP048352, AIP089343, AIP007305, AIP033189, AIP063641, AIP087760, AIP097873, AIP056374. In specific embodiments the composition comprises a combination of strains AIP044543 and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP090377, AIP048352, AIP089343, AIP007305, AIP033189, AIP063641, AIP087760, AIP097873, AIP056374. In specific embodiments the composition comprises a combination of strains AIP090377 and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP048352, AIP089343, AIP007305, AIP033189, AIP063641, AIP087760, AIP097873, AIP056374. In specific embodiments the composition comprises a combination of strains AIP048352 and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP089343, AIP007305, AIP033189, AIP063641, AIP087760, AIP097873, AIP056374. In specific embodiments the composition comprises a combination of strains AIP089343 and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP007305, AIP033189, AIP063641, AIP087760, AIP097873, AIP056374. In specific embodiments the composition comprises a combination of strains AIP007305 and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP033189, AIP063641, AIP087760, AIP097873, AIP056374. In specific embodiments the composition comprises a combination of strains AIP033189, and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP063641, AIP087760, AIP097873, AIP056374. In specific embodiments the composition comprises a combination of strains AIP063641, and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP087760, AIP097873, AIP056374. In specific embodiments the composition comprises a combination of strains AIP087760, and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP097873, AIP056374. In specific embodiments the composition comprises a combination of strains AIP097873, and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP056374. In specific embodiments the composition comprises a combination of strains AIP056374, and one or more of any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760.

In some embodiments, at least one bacterial strain is cultivated in nutrient medium using methods known in the art. The bacterial strain can be cultivated by shake flask cultivation or by small scale or large scale fermentation (including but not limited to continuous, batch, fed-batch, or solid state fermentation) in laboratory or industrial fermenters performed in a suitable medium and under conditions allowing for bacterial cell growth. The cultivation can take place in suitable nutrient medium comprising carbon and nitrogen sources and inorganic salts, using procedures known in the art. Suitable media are available from commercial sources or are prepared according to publications well-known in the art.

Following cultivation, compounds, metabolites, and/or compositions can be extracted from the culture broth. The extract can be fractionated by chromatography. The extract can be further purified using methods well-known in the art. The extract can also be diluted using methods well-known in the art.

The compositions comprising at least a cell of a bacterial strain or cells from a combination of bacterial strains(i.e., at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374 or an active variant of any thereof, or a spore or a forespore or a combination of cells, forespores and/or spores, and/or a composition derived from any one of or a combination of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374, or an active variant of any thereof) can further comprise a suitable carrier and/or typical feed ingredients or combinations thereof. Suitable carriers are insert formulation ingredients added to improve recovery, efficacy, or physical properties and/or to aid in packaging and administration. Such carriers may be added individually or in combination. Carriers are further described elsewhere herein.

A. Active Variants of a Bacterial Strain

Further provided are active variants of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374. Active variants of the various bacterial strains provided herein include, for example, any isolate or mutant of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374 that retain the ability to control one or more undesirable microbes and/or aid in the digestion of some types of feed. An active variant includes a strain having all of the identifying characteristics of the recited strain. A “strain of the invention” includes active variants thereof

The term “mutant” refers to a variant of the parental strain as well as methods for obtaining a mutant or variant in which the antimicrobial or digestive activity is greater than that expressed by the parental strain. The “parent strain” is the original strain before mutagenesis. To obtain such mutants the parental strain may be treated with a chemical such as N-methyl-N′-nitro-N-nitrosoguanidine, ethylmethanesulfone (EMS), or by irradiation using gamma, x-ray, or UV-irradiation, or by other means well known in the art.

In some embodiments, the active variant contains at least one mutation in at least one gene, relative to the deposited strain. The gene(s) may have a role in, for example, biofilm formation, motility, chemotaxis, extracellular secretion, transport (for example ABC transporter proteins), stress responses, volatiles, transcription (for example alternative sigma factors and global transcription regulators), gut colonization, ability to increase the growth of beneficial bacteria in an animal gut, and/or secondary metabolism including synthesis of lipopeptides, polyketides, macromolecular hydrolases (for example proteases and/or carbohydrases), and/or antimicrobial compounds including antibiotics. Secondary metabolism refers to both non-ribosomal and ribosomal synthesis of antimicrobial compounds, including cyclic lipopeptides, polyketides, iturins, bacteriocins (for example plantazolicin and amylocyclicin) and dipeptides (for example bacilysin).

Further active variants of the various bacteria provided herein can be identified by employing, for example, methods that determine the sequence identity relatedness between the 16S ribosomal RNA, methods to identify groups of derived and functionally identical or nearly identical strains include Multi-locus sequence typing (MLST), concatenated shared genes trees, Whole Genome Alignment (WGA), Average Nucleotide Identity, and MinHash (Mash) distance metric.

In one aspect, the active variants of the bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374 include strains that are closely related to any of the disclosed strains by employing the Bishop MLST method of organism classification as defined in Bishop et al. (2009) BMC Biology 7(1)1741-7007-7-3. Thus, in specific embodiments, an active variant of a bacterial strain disclosed herein includes a bacterial strain that falls within at least an 80%, 85%, 86%, 87%, 88%, 89%, 90%, 91%, 92%, 93%. 94%, 95%, 96%, 97%, 98%, 98.5%, 98.8%, 99%, 99.1%, 99.2%, 99.3%, 99.4%, 99.5%, 99.6%, 99.7%, 99.8%, or 99.9% sequence cut off employing the Bishop method of organism classification as set forth in Bishop et al. (2009) BMC Biology 7(1)1741-7007-7-3, which is herein incorporated by reference in its entirety. Active variants of the bacteria identified by such methods will retain the ability to kill or control at least one undesirable microbe and/or to improve digestion when administered in an effective amount to an animal.

In another aspect, the active variant of the bacterial strain(s) disclosed herein include strains that are closely related to any of the disclosed strains on the basis of the Average Nucleotide Identity (ANI) method of organism classification. ANI (see, for example, Konstantinidis, K. T., et al., (2005) PNAS USA 102(7):2567-72; and Richter, M., et al., (2009) PNAS 106(45):19126-31) and variants (see, for example, Varghese, N.J., et al., Nucleic Acids Research (Jul. 6, 2015): gkv657) are based on summarizing the average nucleotides shared between the genomes of strains that align in WGAs. Thus, in specific embodiments, an active variant of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374 disclosed herein includes a bacterial strain that falls within at least a 90%, 95%, 96%, 97%, 97.5%, 98%, 98.5%, 98.8%, 99%, 99.5%, or 99.8% sequence cut off employing the ANI method of organism classification as set forth in Konstantinidis, K.T., et al., (2005) PNAS USA 102(7):2567-72, which is herein incorporated by reference in its entirety. Active variants of the bacteria identified by such methods will retain the ability to kill or control at least one undesirable microbe and/or to improve digestion when administered in an effective amount to an animal.

In another aspect, the active variants of the isolated bacterial strain(s) disclosed herein include strains) that are closely related to any of the above strains (for example, closely related to AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374) on the, basis of 16S rDNA sequence identity. See Stackebrandt. E. et al., “Report of the ad hoc committee for the re-evaluation of the species definition in bacteriology,” Int J Syst Evol Microbiol 52(3):1043-7 (2002) regarding use of 16S rDNA sequence identity for determining relatedness in bacteria. In an embodiment, the active variant is at least 95% to any of the above strains on the basis of 16S rDNA. sequence identity, at least 96% identical to any of the above strains on the basis of 16S rDNA sequence identity, at least 97% identical to any of the above strains on the basis of 16S rDNA sequence identity, at least 98% to any of the above strains on the basis of 16S rDNA sequence identity, at least 98.5% identical to any of the above strains on the basis of 16S rDNA sequence identity, at least 99% identical to any of the above strains on the basis of 16S rDNA sequence identity, at least 99.5% to any of the above strains on the basis of 16S rDNA sequence identity or at least 100% to any of the above strains on the basis of 16S rDNA sequence identity. Active variants of the bacteria identified by such methods will retain the ability to control at least one undesirable microbe, such as a pathogenic bacterium and/or to improve digestion when administered in an effective amount to an animal.

The MinHash (Mash) distance metric is a comparison method that defines thresholds for hierarchical classification of microorganisms at high resolution and requires few parameters and steps (Ondov et al. (2016) Genome Biology 17:132). The Mash distance estimates the mutation rate between two sequences directly from their MinHash sketches (Ondov et al. (2016) Genome Biology 17:132). Mash distance strongly corresponds to Average Nucleotide Identity method (ANI) for hierarchical classification (See, Konstantinidis, K. T. et al. (2005) PNAS USA 102(7):2567-72, herein incorporated by reference in its entirety). That is, an ANI of 97% is approximately equal to a Mash distance of 0.03, such that values put forth as useful classification thresholds in the ANI literature can be directly applied with the Mash distance.

Active variants of the bacterial strain(s) disclosed herein include strains that are closely related to AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374 on the basis of the Minhash (Mash) distance between complete genome DNA sequences. Thus, in specific embodiments, an active variant of a bacterial strain disclosed herein includes bacterial strains having a genome within a Mash distance of less than about 0.015 to the disclosed strains.

In other embodiments, an active variant of a bacterial strain disclosed herein includes a distance metric of less than about 0.001, 0.0025, 0.005, 0.010, 0.015, 0.020, 0.025, or 0.030. A genome, as it relates to the Mash distance includes both bacterial chromosomal DNA and bacterial plasmid DNA. In other embodiments, the active variant of a bacterial strain has a genome that is above a Mash distance threshold to the disclosed strains that is greater than dissimilarity caused by technical variance. In further instances, the active variant of a bacterial strain has a genome that is above a Mash distance threshold to the disclosed strains that is greater than dissimilarity caused by technical variance and has a Mash distance of less than about 0.015. In other instances, the active variant of a bacterial strain has a genome that is above a Mash distance threshold to the disclosed strains that is greater than dissimilarity caused by technical variance and has a Mash distance of less than about 0.001, 0.0025, 0.005, 0.010, 0.015, 0.020, 0.025, or 0.030.

As used herein, “above technical variation” means above the Mash distance between two strains caused by errors in the genome assemblies provided the genomes being compared were each DNA sequenced with at least 20× coverage with the Illumina HiSeq 2500 DNA sequencing technology and the genomes are at least 99% complete with evidence for contamination of less than 2%. While 20× coverage is an art recognized term, for clarity, an example of 20× coverage is as follows: for a genome size of 5 megabases (MB), 100 MB of DNA sequencing from the given genome is required to have 20× sequencing coverage on average at each position along the genome. There are many suitable collections of marker genes to use for genome completeness calculations including the sets found in Campbell et al. (2013) PNAS USA 110(14):5540-45, Dupont et al. (2012) ISMEJ 6:1625-1628, and the CheckM framework (Parks et al. (2015) Genome Research 25:1043-1055); each of these references is herein incorporated in their entirety. Contamination is defined as the percentage of typically single copy marker genes that are found in multiple copies in the given genome sequence (e.g. Parks et al. (2015) Genome Research 25:1043-1055); each of these references is herein incorporated in their entirety. Completeness and contamination are calculated using the same collection of marker genes. Unless otherwise stated, the set of collection markers employed in the completeness and contamination assay is those set forth in Campbell et al. (2013) PNAS USA 110(14):5540-45, herein incorporated by reference.

Exemplary steps to obtain a distance estimate between the genomes in question are as follows: (1) Genomes of sufficient quality for comparison must be produced. A genome of sufficient quality is defined as a genome assembly created with enough DNA sequence to amount to at least 20× genome coverage using Illumina HiSeq 2500 technology. The genome must be at least 99% complete with contamination of less than 2% to be compared to the claimed microbe's genome. (2) Genomes are to be compared using the Minhash workflow as demonstrated in Ondov et al. (2016) Genome Biology 17:132, herein incorporated by reference in its entirety. Unless otherwise stated, parameters employed are as follows: “sketch” size of 1000, and “k-mer length” of 21. (3) Confirm that the Mash distance between the two genomes is less than 0.001, 0.0025, 0.005, 0.010, 0.015, 0.020, 0.025, or 0.030. Using the parameters and methods stated above, a Mash distance of 0.015 between two genomes means the expected mutation rate is 0.015 mutations per homologous position. Active variants of the bacteria identified by such methods will retain the ability to control at least one undesirable microbe, such as a pathogenic bacterium, and/or to improve digestion when administered in an effective amount to an animal.

### III. Formulations

The bacterial strains and combinations of strains provided herein (i.e., cells of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or active variants of any thereof, or a spore or a forespore or a combination of cells, forespores and/or spores, and/or a composition derived from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof), or a supernatant, fermentation product, filtrate, or extract derived therefrom can be formulated as a cell paste, wettable powder, a wettable granule, a cell pellet, dust, granule, a slurry, a dry powder, aqueous or oil based liquid products, gel, and the like. In some embodiments, a composition of the invention is formulated as a spray dried formulation, a wettable granule formulation, a pelleted formulation, or a stable formulation. Common probiotic preparations are liquid solutions and concentrates or lyophilized powders for resuspension, which can be enclosed in a capsule, vial, or pouch. Such formulations will comprise the bacteria provided herein (or combination thereof) or an active variant thereof, and/or a composition derived therefrom, in addition to carriers and other agents. The formulations can be used in a variety of methods as disclosed elsewhere herein.

In some embodiments, feed and/or food compositions can be prepared by combining a formulated bacterial strain of the invention with typical animal feed and/or food ingredients. A formulated bacterial strain of the invention can be used for the preparation of animal feed and pharmaceutical compositions, and/or may be added to drinking and/or rearing water. In other embodiments, the compositions of the present invention are feed additives that are added to an animal's feed or drinking water prior to feeding.

As used herein, “animal feed” includes any animal feed blend known in the art, including rapeseed meal, cottonseed meal, soybean meal, cornmeal, barley, wheat, silage, and haylage.

The bacterial strains disclosed herein and the active variants thereof can be formulated to include at least one carrier, such as proteins, carbohydrates, fats, enzymes, vitamins, immune modulators, oligosaccharides, milk replacers, minerals, amino acids, coccidiostats, acid-based products, medicines (such as antibiotics), other probiotics, and/or prebiotics. Common carriers include cellulose, sugar, glucose, lactose, whey powder, or rice hulls. Carriers can be naturally occurring or non-naturally occurring and can naturally be found with the bacterial strain or not be naturally-occurring with the bacterial strains. The carrier(s) may comprise about 30% weight per weight, weight per volume, or volume per volume, of the final composition. In some embodiments, the carrier(s) may comprise about 40%, about 50%, about 60%, about 70%, about 80%, about 90%, about 91%, about 92%, about 93%, about 94%, about 95%, about 96%, about 97%, about 98%, about 98.5%, about 99.0%, about 99.5%, or about 99.9% weight per weight, weight per volume, or volume per volume of the final composition.

Protein-containing components that can be added to the formulations include soy protein, pea protein, wheat gluten, corn gluten, and combinations thereof. Carbohydrate-containing components include forage, roughage, wheat meal, sunflower meal, soy meal, and combinations thereof. Fat-containing components include oils of animal and/or plant origin, including vegetable oils such as soybean oil, rapeseed oil, sunflower seed oil, flaxseed oil, palm oil, fish oil, and combinations thereof. Additionally, protein-containing components which also contain fats include fish meal, krill meal, bivalve meal, squid meal, shrimp shells, and combinations thereof.

Compositions of the invention may include or be administered with (either at the same time or at different times): enzymes that aid in digestion of feed, such as amylase, glucanase, glucoamylase, cellulase, xylanase, amylase, and/or pectinase; immune modulators, such as antibodies, cytokines, spray-dried plasma; interleukins, and/or interferons; and/or oligosaccharides, such as fructooligosaccharides, mannanoligosaccharides, galactooligosaccharides, inulin, oligofructose enriched inulin, tagatose, and/or polydextrose.

Additional beneficial microbes may be combined with a bacterial strain of the invention into a formulated product. Alternatively, additional formulated probiotics may be combined or mixed with a formulated bacterial strain of the invention, or combination of bacterial strains disclosed herein, into a feed or food composition, or into drinking water, for administration to an animal. Alternatively, the additional probiotic may be administered at a different time. These additional beneficial microbes may be selected from species of Bacillus such as Bacillus subtilis, Bacillus licheniformis, Bacillus lentus, Bacillus pumilus, Bacillus laterosporus, Bacillus coagulans, Bacillus alevi, Bacillus cereus, Bacillus clausii, Bacillus coagulans, Bacillus inaquosorum, Bacillus mojavensis, Bacillus velezensis, Bacillus vallismortis, Bacillus amyloliquefaciens, Bacillus atropheus, Bacillus altitudinis, Bacillus safensis, Bacillus alcalophilus, Bacillus badius, or Bacillus thurigiensis; from species of Enterococcus such as Enterococcus faecium; from species of Clostridium such as Clostridium butyricum; from species of Lactococcus such as Lactococcus lactis or Lactoccus cremoris; from species of Bifidobacterium such as Bifidobacterium adolescentis, Bifidobacterium animalis, Bifidobacterium bifidum, Bifidobacterium infantis, Bifidobacterium longum, Bifidobacterium pseudolongum, or Bifidobacterium thermophilum; from species of Lactobacillus such as Lactobacillus alactosus, Lactobacillus alimentarius, Lactobacillus amylovorans, Lactobacillus amylophilus, Lactobacillus amylovorans, Lactobacillus acidophilus, Lactobacillus agilis, Lactobacillus animalis, Lactobacillus batatas, Lactobacillusbavaricus, Lactobacillus bifermentans, Lactobacillus bidifus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus curvatus, Lactobacillus coprohilus, Lactobacillus delbrueckii, Lactobacillus fermentum, Lactobacillus gasseri, Lactobacillus jugurti, Lactobacillus kefir, Lactobacillus lactis, Lactobacillus leichmannii, Lactobacillus mali, Lactobacillus malefermentans, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus tolerans, Lactobacillus torquens, Lactobacillus ruminis, Lactobacillus sake, Lactobacillus saliverius, Lactobacillus sharpeae, Lactobacillus sobrius, Lactobacillus trichodes, Lactobacillus vaccinostercus, Lactobacillus viridescens, Lactobacillus vitulinus, Lactobacillus xylosus, Lactobacillus yamanashiensis, or Lactobacillus zeae; from species of Megasphaera such as Megasphaera elsdenil; from species of Prevotella such as Prevotella bryantii; from species of Pediococcus such as Pediococcus acidilactici, or Pediococcus pentosaceus; from species of Streptococcus such as Streptococcus cremoris, Streptococcus discetylactis, Streptococcus faecium, Streptococcus lactis, Streptococcus thermophilus, or Streptococcus intermedius; or from species of Propionibacterium such as Propionibacterium freudenreichii, Propionibacterium acidipropionici, Propionibacterium jensenii, Propionibacterium thoenii, Propionibacterium australiense, or Propionibacterium avidum, and/or a combination thereof.

Additional beneficial microbes that may be combined, mixed, or formulated with a strain of the invention also include Bacillus subtilis PB6 (e.g. CLOSTAT® from Kemin, having ATCC Accession NO. PTA-6737 and described in U.S. Pat. No. 7,247,299, incorporated by reference in its entirety herein); B. subtilis C-3102 (e.g. CALSPORIN® from Quality Technology International, having deposit number FERM BP-1096 with the Fermentation Research Institute, Agency of Industrial Science and Technology, in Japan, and described in U.S. Pat. No. 4,919,936, incorporated by reference in its entirety herein); B. subtilis DSM 17299 (e.g. GalliPro® from Christian Hansen), Bacillus licheniformis DSM 17236 (e.g. GalliProTect®), a mixture of B. licheniformis DSMZ 5749 and B. subtilis DSMZ 5750 (e.g. BioPlus®YC from Christian Hansen), B. subtilis DSM 29784 (e.g. Alterion® from Adisseo/Novozymes), Bacillus cereus var. toyoi (e.g. Toyocerin® from Rubinum Animal Health), B. clausii DSM8716, and B. subtilis (e.g. PORCBOOST® from Christian Hansen). In some embodiments, the additional beneficial microbe may be a strain of Lactobacillus spp., such as the MRL1, M35, LA45, LA51, L411, NPC 747, NPC 750, D3, and/or L7 strains. In some embodiments, the additional beneficial microbe may be a strain of Propionibacterium spp., such as the PF24, P5, P63, P1 and/or MRP1 strains. In some embodiments, the additional beneficial microbe may be a microbe which is not a bacterium, such as Saccharamyces cerevisiae, Candida pintolepesii, and/or Aspergillus oryzae. Additional beneficial microbes are known in the art and may be found for example in the 2016 FAO publication “Probiotics in Animal Nutrition — Production, Impact, and Regulation” (Bajagai et al., Ed: Harinder P. S. Makkar, FAO Animal Production and Health Paper No. 179, Rome; incorporated by reference in its entirety herein).

Compositions of the invention may also include prebiotics, which may be combined or mixed with a formulated bacterial strain of the invention into a feed or food composition, or into drinking water, for administration to an animal. Prebiotics are food ingredients that are not readily digestible by enzymes endogenous to the gut (such as those expressed by the animal or those expressed by the resident gut microbiome) and that selectively stimulate the growth and activity of selected groups of intestinal microorganisms that confer beneficial effects upon their host. Typically, it is beneficial microorganism populations that benefit from the presences of prebiotic compounds. Prebiotics can consist of oligosaccharides and other small molecules that serve as metabolic substrates for growth of beneficial microbes. Common prebiotics include galacto-oligosaccharides, fructo-oligosaccharides, inulin, isomalto-oligosaccharies, gentio-oligosaccharides, lactilol, lactosucrose, lactulose, xylosucrose, glycosylsucrose, pyrodextrins, soybean oligosaccharides, guar gum, locust bean gum, arabinan, galactan, pectins, and pectic polysaccharides. While many diverse microbes inhabit the intestinal tract of a host organism, prebiotic compounds are only utilized by the beneficial microbes and lead to a selective enhancement of the beneficial microbe population. A formulation that includes both prebiotics and probiotics may be known as a “synbiotic”.

Preservation of the bacterial strains or combinations of bacterial strains of the invention or variants thereof can include a process of freezing, freeze-drying, and/or spray drying. In some embodiments, the preserved bacteria contain a viable cell concentration of 1×10³ CFU/gram to 1×10¹⁶ CFU/gram, including but not limited to 10³ CFU/gram, 10⁴ CFU/gram, 10⁵ CFU/gram, 10⁶ CFU/gram, 10⁷ CFU/gram, 10⁸ CFU/gram, 10⁹ CFU/gram, 10¹° CFU/gram, 10¹¹ CFU/gram, 10¹² CFU/gram, 10¹³ CFU/gram, 10¹⁴ CFU/gram, 10¹⁵ CFU/gram, and 10¹⁶ CFU/gram. In further embodiments, the viable cell concentration may be about 10³ CFU/gram to about 10¹⁰ CFU/gram, 10³ CFU/gram to about 10⁸ CFU/gram, or 10⁴ CFU/gram to about 10¹⁰ CFU/gram.

Compositions of the invention also include a preservation matrix, which contains and preserves a bacterial culture of a strain of the invention or a variant thereof Such a matrix may include a biologically active binding agent, an antioxidant, a polyol, a carbohydrate, and a proteinaceous material. Such a matrix may be a gel or cream, for example for topical application.

Compositions of the invention include microencapsulation of a bacterial strain of the invention, a variant thereof, a spore or a forespore or a combination of cells, forespores and/or spores, and/or a composition derived therefrom. A bacterial strain of the invention or a variant thereof may be microencapsulated, which may significantly improve cell viability during the freezing and/or drying process. For microencapsulation, the inner core of the microcapsule comprises the bacterial strain(s) of the invention, and the shell is sustained by supporting material. The supporting material may comprise polysaccharides, whey proteins, chitosan, pectin, milk, alginate solutions (for example algae-derived heteropolysaccharides), or collagen. The microcapsule may further comprise cryo-protectants such as glucose, maltodextrin, trehalose, skimmed milk powder, whey protein, or soybean flour.

In some embodiments, a bacterial strain of the invention may be added to feed or drinking water in an amount that is effective to enhance the animal's health and/or performance. In one embodiment, it can be added at an inclusion rate of from about 1×10³ CFU per gram feed or ml drinking water to about 1×10¹⁵ per gram feed or ml drinking water. In another embodiment, it may be added from about 1×10³ CFU per gram feed or ml drinking water to about 1×10⁹ CFU per gram feed or ml drinking water. In yet another embodiment, it may be added from about 1×10⁴ CFU per gram feed or ml drinking water to about 1×10⁸ CFU per gram feed or ml drinking water. In some embodiments, the inclusion rate is about 1×10³ CFU per gram feed or ml drinking water, about 1×10⁴ CFU per gram feed or ml drinking water, about 1×10⁵ CFU per gram feed or ml drinking water, about 1×10⁶ CFU per gram feed or ml drinking water, about 1×10⁷ CFU per gram feed or ml drinking water, about 1×10⁸ CFU per gram feed or ml drinking water, about 1×10⁹ CFU per gram feed or ml drinking water, about 1×10¹⁰ CFU per gram feed or ml drinking water, about 1×10¹¹ CFU per gram feed or ml drinking water, about 1×10¹² CFU per gram feed or ml drinking water, about 1×10¹³ CFU per gram feed or ml drinking water, about 1×10¹⁴ CFU per gram feed or ml drinking water, or about 1×10¹⁵ CFU per gram feed or ml drinking water. In some embodiments, the composition of the invention may need to be diluted to have a CFU count in the above-described ranges upon addition to animal feed or drinking water.

Compositions of the invention can be added to animal feed prior to the pelleting process, such that the composition used forms part of animal feed pellets. In some embodiments, the bacterial strain of the invention, or a variant thereof, is added in spore form to other components of the animal feed prior to the pelleting process. Standard pelleting processes known to those of skill in the art may be used, including extrusion processing of dry or semi-moist feeds. In some embodiments the pelleting process involves temperatures of at least about 65° C. In others, pelleting temperatures are between about 65° C. to about 120° C. In still others, pelleting temperatures are between about 80° C. and about 100° C. In other embodiments, the pelleting temperature is about 60° C., about 65° C., about 70° C., about 75° C., about 80° C., about 85° C., about 90° C., about 95° C., or about 100° C.

The compositions of the present invention may also be administered orally as a pharmaceutical in combination with a pharmaceutically acceptable carrier. Optimal dosage levels for various animals can easily be determined by those skilled in the art, for example by evaluating the composition's ability to (i) inhibit or reduce undesirable microbes, such as pathogenic bacteria, in the gut at various doses; (ii) increase or maintain levels of beneficial bacteria; and/or (iii) enhance animal health at various doses.

In some embodiments, compositions of the invention may be formulated with or added in combination with fish feed. In other embodiments, compositions of the invention may be added to fish-rearing waters. The composition may be added in an amount that is effective to enhance the health of the fish or other aquatic animal, such as shrimp. Such an effective amount in some embodiments may be between about 10³ to about 10¹⁵ CFU per ml of rearing water; between about 10³ to about 10¹² CFU per ml of rearing water; between about 10³ to about 10¹⁰ CFU per ml of rearing water; or between about 10³ to about 10⁸ CFU per ml of rearing water.

The viability of a bacterial strain, such as AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or active variant of any thereof, in a composition or formulation can be quantitated using an epifluorescence assay in which fluorescent dyes that are specific for cells with intact membranes or disrupted membranes are utilized, such as those assays that use a SYTO 9 nucleic acid stain that fluoresces green indicating a cell has an intact membrane and propidium iodide that fluoresces red indicating a cell with a disrupted membrane that is not viable (see, for example, LIVE/DEAD® BacLight™ Bacterial Viability and Counting Kit from Molecular Probes; and Ivanova et al. (2010) Biotechnology &Biotechnological Equipment 24:supl, 567-570). It is known that following desiccation, some bacterial strains, such as for example certain Pseudomonas strains, enter a metabolically active state in which the cells are viable but not culturable (VBNC) (Pazos-Rojas et al. (2019) PLoS ONE 14(7):e0219554). Cells in a VBNC state retain the ability to be cultured if reconstituted, for example, in water or root exudates, when exposed to particular metals or ions, or any other reconstitution method that is specific for the individual VBNC bacterial strain.

In some embodiments, the composition or formulation comprises a concentration (e.g., as measured by viability) of a bacterial strain or a combination of bacterial strains of at least about 10¹ cells/gram to about 10⁶ cells/gram, 10² cells/gram to about 10⁵ cells/gram, 10² cells/gram to about 10⁴ cells/gram, 10³ cells/gram to about 10⁶ cells/gram, 10⁴ cells/gram to about 10⁸ cells/gram, at least about 10⁵ cells/gram to about 10¹¹ cells/gram, about 10⁷ cells/gram to about 10¹⁰ cells/gram, about 10⁷ cells/gram to about 10¹¹ cells/gram, about 10⁶ cells/gram to about 10¹⁰ cells/gram, about 10⁶ cells/gram to about 10¹¹ cells/gram, about 10¹¹ cells/gram to about 10¹² cells/gram, about 10⁵cells/gram to about 10¹⁰ cells/gram, about 10⁵ cells/gram to about 10¹² cells/gram, about 10⁵ cells/gram to about 10⁶ cells/gram, about 10⁶ cells/gram to about 10⁷ cells/gram, about 10⁷ cells/gram to about 10⁸ cells/gram, about 10⁸ cells/gram to about 10⁹ cells/gram, about 10⁹ cells/gram to about 10¹⁰ cells/gram, about 10¹⁰ cells/gram to about 10¹¹ cells/gram, or about 10¹¹ cells/gram to about 10¹² cells/gram. In some embodiments, the concentration of the bacterial strain comprises at least about 10² cells/gram, at least about 10³ cells/gram, at least about 10⁴ cells/gram, at least about 10⁵ cells/gram, at least about 10⁶ cells/gram, at least about 10⁷ cells/gram, at least about 10⁸ cells/gram, at least about 10⁹ cells/gram, at least about 10¹⁰ cells/gram, at least about 10¹¹ cells/gram, at least about 10¹² cells/gram, or at least about 10¹³ cells/gram of viable cells as measured with an epifluorescence assay.

In liquid compositions and formulations, the amount of a bacterial strain or a combination of bacterial strains of the invention or active variants thereof, disclosed herein can comprise a concentration of at least about 10¹ cells/mL to about 10⁶ cells/mL, 10² cells/mL to about 10⁵ cells/mL, 10² cells/mL to about 10⁴ cells/mL, 10³ cells/mL to about 10⁶ cells/mL, 10⁴ cells/mL to about 10⁸ cells/mL, at least about 10³ to about 10⁹ cells/mL, at least about 10³ to about 10⁶ cells/mL, at least about 10⁴ to about 10¹¹ cells/mL, at least about 10⁵ cells/mL to about 10¹¹cells/mL, about 10⁵ cells/mL to about 10¹⁰ cells/mL, about 10⁵ cells/mL to about 10¹² cells/mL, about 10⁵ cells/mL to about 10⁶ cells/mL, about 10⁶ cells/mL to about 10⁷ cells/mL, about 10⁷ cells/mL to about 10⁸ cells/mL, about 10⁸ cells/mL to about 10⁹ cells/mL, about 10⁹ cells/mL to about 10¹⁰ cells/mL, about 10¹⁰ cells/mL to about 10¹¹ cells/mL, or about 10¹¹ cells/mL to about 10¹² cells/mL or at least about 10³ cells/mL, at least about 10⁴ cells/mL, at least about 10⁵cells/mL, at least about 10⁶ cells/mL, at least about 10⁷ cells/mL, at least about 10⁸ cells/mL, at least about 10⁹ cells/mL, at least about 10¹⁰ cells/mL, at least about 10¹¹ cells/mL, at least about 10¹² cells/mL of viable cells as measured with an epifluorescence assay.

In still other embodiments, the concentration of a metabolite within a composition or formulation comprising a bacterial strain or a combination of bacterial strains comprising at least one strains selected from AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or active variant of any thereof, can be measured as a surrogate of the viability and/or activity of the bacterial strain in the composition or formulation. Said metabolite may be used as a reporter metabolite for anti-microbial activity, and further may be co-regulated with other anti-microbial metabolites that are active in AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or active variants of any thereof. The presence of the reporter metabolite is a measure of intact cells and cell concentration within a composition or formulation. In some embodiments, the reporter metabolite is a lipopeptide or peptidic toxin. In further embodiments, the reporter metabolite is lichenysin, fengycin, plipastatin, bottromycin A2, bacilysin, subtilisin, or mycosubtilin. In some embodiments, the reporter metabolite is retained within cells and not secreted, so measurement first requires cell lysis. The reporter metabolite can then be measured using any analytical chemistry method known in the art, including but not limited to, high performance liquid chromatography with ultraviolet detection (HPLC-UV) of a composition or formulation, such as that described in Hill et al. (1994) Appl Env Micro 60(1) 78-85, which is herein incorporated by reference in its entirety. In some embodiments, the presently disclosed compositions or formulations comprise between about 50 μg/g to 2000 μg/g 75 μg/g to 2000 μg/g, 100 μg/g to 2000 μg/g, 200 μg/g to 1800 μg/g, 300 μg/g to 1500 μg/g, 300 μg/g to 1300 μg/g, 400 μg/g to 1500 μg/g, 400 μg/g to 1300 μg/g, 300 μg/g to 1000 μg/g, 400 μg/g to 1000 μg/g, 500 μg/g to 1000 μg/g, 500 μg/g to 1300 μg/g, 600 μg/g to 1000 μg/g, 600 μg/g to 1300 μg/g, 600 μg/g to 1500 μg/g, or about 50 μg/g about 75 μg/g, about 100 μg/g, about 200 μg/g, about 300 μg/g, about 400 μg/g, about 500 μg/g, about 600 μg/g, about 700 μg/g, about 800 μg/g, about 900 μg/g, about 1000 μg/g, about 1100 μg/g, about 1200 μg/g, about 1300 μg/g, about 1400 μg/g, about 1500 μg/g, about 1500 μg/g, about 1600 μg/g, about 1700 μg/g, about 1800 μg/g, about 1900 μg/g, and about 2000 μg/g expressed as μg of reporter metabolite per g of bacteria.

### IV. Methods of Use

### A. Methods of Controlling Undesirable Microbes

In some embodiments, undesirable microbes may be controlled by exposing the undesirable microbes to a) an effective amount of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof wherein the active variant comprises a bacterial strain having a genome within a Mash distance of about 0.015; b) an effective amount of at least one of a spore, or a forespore, or a combination of cells, forespores and/or spores from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374 or a combination of or an active variant of any thereof, wherein the active variant comprises a bacterial strain having a genome within a Mash distance of about 0.015; and/or c) an effective amount of a supernatant, fermentation product, filtrate, or extract derived from a whole cell culture of at least one of or a combination selected from bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015.

“Exposing” the undesirable microbe includes directly contacting the microbe with a strain of the invention, and/or introducing a strain of the invention into the same environment as the undesirable microbe, for example the gastrointestinal tract of an animal. By being exposed to a strain of the invention, the undesirable microbe may be controlled by said strain without direct contact by said strain. The “effective amount” is the amount needed to control undesirable microbes. In some embodiments, the effective amount of the bacterial strain or active variant thereof or combination of bacterial strains comprises or consists of at least about 10³ CFU/gram to about 10¹⁵ CFU/gram, at least about 10³ CFU/gram to about 10¹² CFU/gram, at least about 10³ CFU/gram to about 10¹⁰ CFU/gram, at least about 10³ CFU/gram to about 10⁹ CFU/gram, or at least about 10⁴ CFU/gram to about 10⁸ CFU/gram, including but not limited to about 10³ CFU/gram, about 10⁴ CFU/gram, about 10⁵ CFU/gram, about 10⁶ CFU/gram, about 10⁷ CFU/gram, about 10⁸ CFU/gram, about 10⁹ CFU/gram, about 10¹⁰ CFU/gram, about 10¹¹ CFU/gram, about 10¹² CFU/gram, about 10¹³ CFU/gram, about 10¹⁴ CFU/gram, and about 10¹⁵ CFU/gram, or equivalent measure of bacterial concentration. In some embodiments, the effective amount of the bacterial strain or active variant thereof or combination of bacterial strains comprises or consists of at least about 10³ CFU/ml to about 10¹⁵ CFU/ml, at least about 10³ CFU/ml to about 10¹⁰ CFU/ml, at least about 10³ CFU/ml to about 10⁹ CFU/ml, at least about 10³ CFU/ml to about 10⁸ CFU/ml, or at least about 10⁴ CFU/ml to about 10⁸ CFU/ml, including but not limited to about 10³ CFU/ml, about 10⁴ CFU/ml, about 10⁵ CFU/ml, about 10⁶ CFU/ml, about 10⁷ CFU/ml, about 10⁸ CFU/ml, about 10⁹ CFU/ml, about 10¹⁰ CFU/ml, about 10¹¹ CFU/ml, about 10¹² CFU/ml, about 10¹³ CFU/ml, about 10¹⁴ CFU/ml, and about 10¹⁵ CFU/ml, or equivalent measure of bacterial concentration. In some embodiments, the effective amount of at least one of a spore, or a forespore, or a combination of cells, forespores and/or spores from any one of or any combination of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374 or an active variant of any thereof comprises or consists of at least about 10³ CFU/gram to about 10¹⁵ CFU/gram, at least about 10³ CFU/gram to about 10¹¹ CFU/gram, at least about 10³ CFU/gram to about 10¹⁰ CFU/gram, at least about 10³ CFU/gram to about 10⁹ CFU/gram, or at least about 10⁴ CFU/gram to about 10⁸ CFU/gram, including but not limited to about 10³ CFU/gram, about 10⁴ CFU/gram, about 10⁵ CFU/gram, about 10⁶ CFU/gram, about 10⁷ CFU/gram, about 10⁸ CFU/gram, about 10⁹ CFU/gram, about 10¹⁰ CFU/gram, about 10¹¹ CFU/gram, about 10¹² CFU/gram, about 10¹³ CFU/gram, about 10¹⁴ CFU/gram, and about 10¹⁵ CFU/gram or equivalent measure of bacterial concentration. In some embodiments, the effective amount of at least one of a spore, or a forespore, or a combination of cells, forespores and/or spores from any one of or any combination of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374 or an active variant of any thereof comprises or consists of at least about 10³ CFU/ml to about 10¹⁵ CFU/ml, at least about 10³ CFU/ml to about 10¹¹ CFU/ml, at least about 10³ CFU/ml to about 10¹⁰ CFU/ml, at least about 10³ CFU/ml to about 10⁹ CFU/ml, or at least about 10⁴ CFU/ml to about 10⁸ CFU/ml, including but not limited to about 10³ CFU/ml, about 10⁴ CFU/ml, about 10⁵ CFU/ml, about 10⁶ CFU/ml, about 10⁷ CFU/ml, about 10⁸ CFU/ml, about 10⁹ CFU/ml, about 10¹⁰ CFU/ml, about 10¹¹ CFU/ml, about 10¹² CFU/ml, about 10¹³ CFU/ml, about 10¹⁴ CFU/ml, and about 10¹⁵ CFU/ml or equivalent measure of bacterial concentration. In particular embodiments, the effective amount is a measure of the combined concentration of at least two (2) bacterial strains selected from AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374 or an active variant of any thereof.

### B. Methods of Treating or Preventing Disease or Increasing Animal Health

It is known in the art that control of undesirable microbes present in the gastrointestinal tract of an animal by decreasing their ability to grow in the gut of an animal reduces incidence of disease caused by said microbes. Additionally, it is well-known that the administration of beneficial microbes, such as beneficial bacteria, can reduce the incidence of undesirable microbes in animals (see, for example, U.S. Pat. No. 7,063,836, incorporated by reference herein). It is also recognized that the consumption of exogenous bacteria, for example probiotics, can elicit beneficial effects upon a host. For example, in humans, consumption of probiotics has been shown to reduce the risk of antibiotic-associated diarrhea and increase remission rates in adults with ulcerative colitis (Wilkins and Sequoia, American Family Physician (2017), 96(3): 170-179).

It is also well established that the addition of beneficial bacteria to animal feed, for example as probiotics, can improve animal efficiency and health. Healthy microbial populations in the gastrointestinal tract of an animal are often associated with enhanced animal performance, reflecting more efficient digestion and improved immunity. Such improvements may be measured by the weight gain-to-feed intake ratio (feed efficiency), average daily weight gain, average daily feed intake, feed conversion in the animal and disease incidence (see, for example, Liao and Nychoti, Animal Nutrition (2017), 3: 331-343, herein incorporated in its entirety). For dairy cows, improvements may also be measured by milk yield or milk composition (U.S. Pat. Nos. 5,529,793 and 5,534,271, incorporated by reference herein). Increased productivity in farm animals may be measured by production of more or higher quality eggs, milk, or meat, or increased production of weaned offspring.

Provided herein are methods for improving animal health and/or performance by providing an effective amount of at least one bacterial strain provided herein or combination of bacterial strains provided herein or an active variant thereof, and/or a composition derived therefrom, to the animal. A composition of the invention may increase animal health by improving or establishing a healthy microbial population in the gastrointestinal tract of the animal. This improvement can be determined by increased growth rate, increased average weight gain, higher feed intake, improved feed conversion ratio, higher feed conversion efficiency, and improved nutrient digestibility, all of which can be measured by one of skill in the art (see e.g., Liao and Nyachoti, (1997) Animal Nutrition 3: 331-343, and references cited therein). In some embodiments, the term “improvement of animal health” refers to the improvement in a diseased animal's physiological state. In some embodiments, an animal's health (e.g., growth rate, average weight gain, feed intake, feed conversion ratio, feed conversion efficiency, nutrient digestibility) can be improved by at least about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% or more when compared to non-treated control animals.

Also provided herein are methods for controlling undesirable microbes such as pathogenic bacteria, comprising providing to an animal an effective amount of at least one bacterial strain provided herein or an active variant thereof, and/or a composition derived therefrom wherein the bacterial strain and/or the composition derived therefrom controls the undesirable microbes (e.g., pathogenic bacteria). The pathogenic bacteria include, but are not limited to, pathogenic strains of Clostridia spp (such as C. perfringens and C. dificile), Burkeholderia spp. (such as B. pseudomallei), Pseudomonas spp. (such as P. aeruginosa), Acenitobacter spp. (such as A. baumannii), Salmonella spp. (such as S. enterica, S. arizonae, S. typhirium, S. enteritidis, and S. bonglori), Listeria spp. (such as L. moncytogenes, L. seeligeri, and L. welshimeri), Escherichia spp (such as E. coli), Enterococci spp. (such as E. faecalisand E. bonglori), Staphylococci spp. (such as S. aureus), Aeromonas spp, Streptococci spp, Campylobacter spp, Haemophilus spp, Brachyspira spp, and Vibrio spp. Accordingly, administration of an effective amount of a bacterial strain provided herein or a combination of bacterial strains provided herein can reduce the total number or growth rate of a pathogenic bacterial strain by at least about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% or about 10-20%, about 20-30%, about 10-50%, about 30-40%, about 40-50%, about 50-60%, about 60-70%, about 70-80%, about 80-90%, about 90-99%, about 25-50%, about 50-75%, about 75-99%, or more when compared to non-treated control animals.

Also provided herein are methods of reducing susceptibility to pathogenic bacteria and/or increasing resistance to pathogenic bacteria comprising providing to an animal infected with pathogenic bacteria an effective amount of at least one bacterial strain provided herein or an active variant thereof, and/or a composition derived therefrom wherein the bacterial strain and/or the composition derived therefrom controls the pathogenic bacteria. Provided herein are methods of treating or preventing infection by pathogenic bacteria comprising providing to an animal infected with pathogenic bacteria or at risk of developing an infection from a pathogenic bacteria an effective amount of at least one bacterial strain provided herein or an active variant thereof, and/or a composition derived therefrom wherein the bacterial strain or combination of bacterial strains and/or the composition derived therefrom controls the pathogenic bacteria that causes the infection. In certain embodiments, the bacterial strain(s) provided herein or active variant thereof may comprise a cell of at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof; or a spore, or a forespore or a combination of cells, forespores and/or spores from at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof; or a supernatant, fermentation product, filtrate or extract of any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof.

An animal treated with the bacterial strain or combination of bacterial strains provided herein or an active variant thereof may show a reduced severity or reduced development of disease in the presence of pathogenic bacteria by a statistically significant amount. A reduced severity or reduced development of disease or damage can be a reduction of about 10% to about 20%, about 20% to about 30%, about 30% to about 40%, about 40% to about 50%, about 50% to about 60%, about 60% to about 70%, about 70% to about 80%, about 80% to about 90%, or about 90% to about 100% when compared to non-treated control animals. In other instances, the animal treated with a bacterial strain provided herein or an active variant thereof may show a reduced severity or reduced development of disease in the presence of a pathogenic bacteria of at least about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% or about 10-20%, about 20-30%, about 10-50%, about 30-40%, about 40-50%, about 50-60%, about 60-70%, about 70-80%, about 80-90%, about 90-99%, about 25-50%, about 50-75%, about 75-99% greater when compared to non-treated control animals. Methods for assessing animal disease severity are known, and include reductions of any symptom of the disease including but not limited to diarrhea incidence (such as for example the rate of post-weaning diarrhea in mammalian farm animals), mortality (such as for example pre- or post-weaning mortality in mammalian farm animals), weight loss, reduction of growth rate, reduction of milk production, inflammation, loss of pregnancy, skin lesions, coughing, sneezing, diarrhea, increased body temperature, or a combination thereof

In specific embodiments, the bacterial strains, active variants thereof, and/or a composition derived therefrom provided herein reduce the disease or disease symptoms resulting from a pathogenic bacteria or other undesirable microbe by a statistically significant amount, including for example, at least about 10% to at least about 20%, at least about 20% to about 50%, at least about 10% to about 60%, at least about 30% to about 70%, at least about 40% to about 80%, or at least about 50% to about 90% or greater. Hence, the methods of the invention can be utilized to protect animals from disease or disease symptoms caused by undesirable microbes including pathogenic bacteria.

The term “treat” or “treating” or its derivatives includes substantially inhibiting, slowing, or reversing the progression of a condition, substantially ameliorating symptoms of a condition or substantially preventing the appearance of symptoms or conditions brought about by the pathogenic bacteria. In specific embodiments, treating a condition comprises reducing the severity or delaying the onset of at least one symptom of the condition.

In particular embodiments, “controlling” and “protecting” an animal from an undesirable microbe refers to one or more of inhibiting or reducing the growth, germination, reproduction, and/or proliferation of an undesirable microbe; and/or killing, removing, destroying, or otherwise diminishing the occurrence, and/or activity of an undesirable microbe. As such, an animal treated with the bacterial strain provided herein, a variant thereof, and/or a composition derived therefrom may show a reduced severity or reduced development of disease in the presence of an undesirable microbe by a statistically significant amount. Alternatively, the animal treated with a bacterial strain provided herein, a variant thereof, and/or a composition derived therefrom may exhibit increased signs of animal healthiness. Signs of healthiness include improvements in animal performance, which may be measured by the weight gain-to-feed intake ratio (feed efficiency), average daily weight gain, average daily feed intake, feed conversion in the animal and disease incidence. Signs of healthiness also include reduction of mortality within a population of animals.

The term “prevent” and its variations means the countering in advance of pathogenic microbe infection, growth, or proliferation. In some embodiments, the composition is applied before exposure to pathogenic bacteria. The term “inhibit” and all variations of this term is intended to encompass the restriction or prohibition of infection, growth, and/or proliferation by a pathogenic microbe, for example pathogenic bacteria.

The terms “delay”, “retard” and all variations thereof are intended to encompass the slowing of the progress of infection, growth, and/or proliferation by a pathogenic microbe. The expression “delaying the onset” is interpreted as preventing or slowing the progression of infection, growth, and/or proliferation of a pathogenic microbe for a period of time, such that said pathogenic microbe infection, growth, and/or proliferation do not progress as far along in development, or appear later than in the absence of the treatment according to the invention.

The terms “ameliorate” and “amelioration” relate to the improvement in the treated animal condition brought about by the compositions and methods provided herein.

Administration of the strains of the invention or variants thereof or compositions derived therefrom may also be used to restore or maintain intestinal microbial balance before, during, or after administration of therapeutic amounts of antibiotics by inhibiting growth of pathogenic microbes and/or increasing or maintaining growth of beneficial microbes. In some embodiments, “therapeutic amount” refers to an amount sufficient to ameliorate or reverse a disease state in an animal.

Compositions of the invention can be administered to an animal in a multitude of ways well-known in the art. Most typically, compositions of the invention are provided to the animal orally, as a feed additive or probiotic. In some embodiments, a composition of the invention is added to animal feed, either prior to the pelleting process, so that the composition is part of the animal feed formulation, or after the pelleting process, so that the composition is added to the feed separately but prior to providing to the animal. In other embodiments, compositions of the invention are added to the drinking water that is provided to the animal. In other embodiments, compositions of the invention are provided to the animal through means other than oral administration. In some embodiments, compositions of the invention are administered to an animal as an injection. In other embodiments, compositions of the invention are administered to an animal as a topical application. In some embodiments, compositions of the inventions may be added to rearing waters, as in the case of aquatic animals, so that the animals are exposed to compositions of the invention in their aquatic environments.

The compositions and methods of the invention may be provided and/or administered to any animal, including vertebrates such as mammals (including humans), reptiles, birds, and aquatic animals including fish and crustaceans. Animals that may be treated with a composition of the invention include farm animals, companion animals, and animals used for sports, recreation, or work. This includes horses, dogs, cats, birds, exotic pets including reptiles, and zoo animals. Animals include monogastric animals such as horses, humans, pigs, and birds. Birds include poultry such as chicken, turkey, duck, geese, guinea fowl, and also ostrich, emu, and also game birds such as quail, chukar, pheasant, grouse, Cornish hens, and partridge. Chickens refer to broiler chickens and egg-producing chickens. Animals include polygastric animals, also referred to as ruminants, such as cattle, sheep, goats, camels, llama, alpacas, bison, buffalo, deer, wildebeest, and antelope. In some embodiments, the animals are preruminants, which are ruminants ranging in age from birth to about 12 weeks, such as calves. The compositions of the present invention may be administered to young weaning animals, such as preruminants or weaning piglets, in conjunction with milk replacers. Milk replacers refer to formulated feed intended to replace colostrum during milk feed stages of the preruminant or weaning animal. Aquatic animals include fish such as salmon, trout, catfish, tilapia, flounder, or ornamental fish, and also crustaceans such as shrimp, crab, or lobster.

Non-limiting embodiments of the invention include:

1. A composition comprising:
(a) at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015, and wherein said bacterial strain or an active variant thereof is present at about 10³ CFU/gram to about 10¹² CFU/gram or at about 10³ CFU/ml to about 10¹² CFU/ml;
(b) at least one of a spore, or a forespore, or a combination of cells, forespores, and/or spores from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015, and wherein said spore, forespore, or a combination of cells, forespores, and/or spores or an active variant thereof is present at about 10³ CFU/gram to about 10¹² CFU/gram or about 10³ CFU/ml to about 10¹² CFU/ml; and/or
(c) a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

wherein an effective amount of said composition controls the growth of at least one undesirable microbe.

2. The composition of embodiment 1, wherein said composition comprises at least one of a cell, a spore, or a forespore, or a combination of cells, forespores, and/or spores of AIP004816, or an active variant thereof, and/or AIP053802, or an active variant thereof, and/or AIP006035, or an active variant thereof, wherein the active variant comprises a bacterial strain having a genome within a Mash distance of about 0.015.

3. The composition of embodiment 2, wherein said composition further comprises at least one of a cell, a spore, or a forespore, or a combination of cells, forespores, and/or spores of AIP088262, or an active variant thereof, and/or AIP097873, or an active variant thereof, wherein the active variant comprises a bacterial strain having a genome within a Mash distance of about 0.015.

4. The composition of embodiment 1, wherein said composition comprises at least one of a cell, a spore, or a forespore, or a combination of cells, forespores, and/or spores of AIP004816, or an active variant thereof, and AIP012656, or an active variant thereof wherein the active variant comprises a bacterial strain having a genome within a Mash distance of about 0.015.

5. The composition of embodiment 1, wherein said composition comprises at least one of a cell, a spore, or a forespore, or a combination of cells, forespores, and/or spores of AIP088262, or an active variant thereof, and/or AIP004816, or an active variant thereof, and/or AIP053802, or an active variant thereof, wherein the active variant comprises a bacterial strain having a genome within a Mash distance of about 0.015.

6. The composition of embodiment 5, wherein said composition further comprises at least one of a cell, a spore, or a forespore, or a combination of cells, forespores, and/or spores of AIP006035, or an active variant thereof, and/or AIP022568, or an active variant thereof, wherein the active variant comprises a bacterial strain having a genome within a Mash distance of about 0.015.

7. The composition of embodiment 1, wherein said composition comprises at least one of a cell, a spore, or a forespore, or a combination of cells, forespores, and/or spores of AIP088262, or an active variant thereof, and/or AIP053802, or an active variant thereof, and/or AIP006035, or an active variant thereof wherein the active variant comprises a bacterial strain having a genome within a Mash distance of about 0.015.

8. The composition of embodiment 7, wherein said composition further comprises at least one of a cell, a spore, or a forespore, or a combination of cells, forespores, and/or spores of AIP087760, or an active variant thereof, wherein the active variant comprises a bacterial strain having a genome within a Mash distance of about 0.015.

9. The composition of embodiment 1, wherein said composition comprises a combination of at least one of a cell, a spore, or a forespore, or a combination of cells, forespores, and/or spores of at least two bacterial strains selected from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant thereof, wherein the active variant comprises a bacterial strain having a genome within a Mash distance of about 0.015.

10. The composition of embodiment 1, wherein said composition comprises a combination of at least one of a cell, a spore, or a forespore, or a combination of cells, forespores, and/or spores of at least three bacterial strains selected from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant thereof, wherein the active variant comprises a bacterial strain having a genome within a Mash distance of about 0.015.

11. The composition of embodiment 1, wherein said composition comprises a combination of at least one of a cell, a spore, or a forespore, or a combination of cells, forespores, and/or spores of at least four bacterial strains selected from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant thereof, wherein the active variant comprises a bacterial strain having a genome within a Mash distance of about 0.015.

12. The composition of embodiment 1, wherein said composition comprises a combination of at least one of a cell, a spore, or a forespore, or a combination of cells, forespores, and/or spores of at least five bacterial strains selected from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant thereof, wherein the active variant comprises a bacterial strain having a genome within a Mash distance of about 0.015.

13. The composition of any one of embodiments 1 to 12, wherein said bacterial strain, spore, forespore, or combination of cells, forespores, and/or spores, or the active variant thereof is present at about 10³ CFU/gram to about 10¹⁰ CFU/gram or at about 10³ CFU/ml to about 10¹⁰ CFU/ml.

14. The composition of any one of embodiments 1 to 13, wherein said bacterial strain, spore, forespore, or combination of cells, forespores, and/or spores, or the active variant thereof is present at about 10⁴ CFU/gram to about 10⁸ CFU/gram or at about 10⁴ CFU/ml to about 10⁸ CFU/ml.

15. The composition of any of embodiments 1 to 14, wherein said composition comprises a cell paste, a wettable powder, a spray dried formulation, a wettable granule formulation, a pelleted formulation, or a stable formulation.

16. The composition of any of embodiments 1 to 15, wherein said composition further comprises at least one or more of a carrier, proteins, carbohydrates, fats, other probiotics, prebiotics, enzymes, vitamins, immune modulators, milk replacers, minerals, amino acids, coccidiostats, acid-based products, and medicines.

17. The composition of any of embodiments 1 to 16, wherein said composition comprises at least one additional bacterial strain.

18. The composition of embodiment 17, wherein the additional bacterial strain is selected from the group consisting of a Bacillus spp, Enterococcus spp., Bifidobacterium spp., Lactobacillus spp., Streptococcus spp., Propionibacterium spp., Megasphaera spp., Prevotella spp.,and a Pediococcus spp.

19. The composition of embodiment 17, wherein said additional bacteria strain is selected from the group consisting of Bacillus subtilis, Bacillus licheniformis, Bacillus lentus, Bacillus pumilus, Bacillus laterosporus, Bacillus coagulans, Bacillus alevi, Bacillus cereus, Bacillus clausii, Bacillus coagulans, Bacillus mojavensis, Bacillus velezensis Bacillus vallismortis, Bacillus amyloliquefaciens, Bacillus atropheus, Bacillus altitudinis, Bacillus inaquosorum, Bacillus safensis, Bacillus alcalophilus, Bacillus badius, Bacillus thurigiensis, Bifidobacterium adolescentis, Bifidobacterium animalis, Bifidobacterium bifidum, Bifidobacterium infantis, Bifidobacterium longum, Bifidobacterium pseudolongum, Bifidobacterium thermophilum, Lactobacillus alactosus, Lactobacillus alimentarius, Lactobacillus amylovorans, Lactobacillus amylophilus, Lactobacillus amylovorans, Lactobacillus acidophilus, Lactobacillus agilis, Lactobacillus animalis, Lactobacillus batatas, Lactobacillusbavaricus, Lactobacillus bifermentans, Lactobacillus bidifus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus curvatus, Lactobacillus coprohilus, Lactobacillus delbrueckii, Lactobacillus fermentum, Lactobacillus gasseri, Lactobacillus jugurti, Lactobacillus kefir, Lactobacillus lactis, Lactobacillus leichmannii, Lactobacillus mali, Lactobacillus malefermentans, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus tolerans, Lactobacillus torquens, Lactobacillus ruminis, Lactobacillus sake, Lactobacillus saliverius, Lactobacillus sharpeae, Lactobacillus sobrius, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Streptococcus cremoris, Streptococcus discetylactis, Streptococcus faecium, Streptococcus lactis, Streptococcus thermophilus, Streptococcus intermedius, Enterococcus faecium, Propionibacterium freudenreichii, Propionibacterium acidipropionici, Propionibacterium jensenii, Propionibacterium thoenii, Propionibacterium australiense, Propionibacterium avidum, Megasphaera elsdenil, Prevotella bryantii, and Pediococcus acidilactici.

20. The composition of embodiment 17, wherein said composition further comprises at least one of bacterial strain Bacillus subtilis PB6, B. subtilis C-3102, B. subtilis DSM 17299, Bacillus licheniformis DSM 17236, B. licheniformis DSMZ 5749, B. subtilis DSMZ 5750, B. subtilis DSM 29784, or an active variant of any thereof.

21. The composition of any of embodiments 1-20, wherein said undesirable microbe comprises at least one pathogenic bacteria.

22. The composition of embodiment 21, wherein said pathogenic bacteria comprises a strain selected from the group consisting of Clostridia spp. (such as C. perfringens and C. dificile), Burkeholderia spp. (such as B. pseudomallei), Pseudomonas spp. (such as P. aeruginosa), Acenitobacter spp. (such as A. baumannii), Salmonella spp (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

23. A composition comprising a spray dried formulation, comprising:

(a) at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015; and/or,

(b) at least one of a spore, or a forespore, or a combination of cells, forespores and/or spores from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

(c) a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

wherein an effective amount of said composition controls the growth of at least one undesirable microbe.

24. A composition comprising a wettable power, comprising:

(a) at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015, and wherein said bacterial strain or an active variant thereof is present at about 10³ CFU/gram to about 10¹² CFU/gram or at about 10³ CFU/ml to about 10¹² CFU/ml;

(b) at least one of a spore, or a forespore, or a combination of cells, forespores, and/or spores from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015, and wherein said spore, forespore, or a combination of cells, forespores, and/or spores or an active variant thereof is present at about 10³ CFU/gram to about 10¹² CFU/gram or at about 10³ CFU/ml to about 10¹² CFU/ml; and/or

(c) a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

wherein an effective amount of said composition controls the growth of at least one undesirable microbe.

25. An isolated biologically pure culture of a bacterial strain comprising:

(a) AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015, and wherein said bacterial strain or an active variant thereof is present at about 10³ CFU/gram to about 10¹² CFU/gram or at about 10³ CFU/ml to about 10¹² CFU/ml; or,

(b) a spore, or a forespore, or a combination of cells, forespores and/or spores from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015, and wherein said spore, forespore, or a combination of cells, forespores, and/or spores or an active variant thereof is present at about 10³ CFU/gram to about 10¹² CFU/gram or at about 10³ CFU/ml to about 10¹² CFU/ml;

wherein an effective amount of said culture controls the growth of at least one undesirable microbe. 26. The isolated biologically pure culture of embodiment 25, wherein said undesirable microbe comprises at least one pathogenic bacteria.

27. The isolated biologically pure culture of embodiment 26, wherein said pathogenic bacteria is a strain selected from the group consisting of Clostridia spp. (such as C. perfringens and C. dificile), Burkeholderia spp. (such as B. pseudomallei), Pseudomonas spp. (such as P. aeruginosa), Acenitobacter spp. (such as A. baumannii), Salmonella spp (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

28. A bacterial culture grown from:

(a) AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015; or,

(b) a spore, or a forespore, or a combination of cells, forespores and/or spores from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

wherein said bacterial culture controls the growth of at least one undesirable microbe.

29. The bacterial culture of embodiment 28, wherein said undesirable microbe comprises at least one pathogenic bacteria.

30. The bacterial culture of embodiment 29, wherein said pathogenic bacteria comprises a strain selected from the group consisting of Clostridia spp. (such as C. perfringens and C. dificile), Burkeholderia spp. (such as B. pseudomallei), Pseudomonas spp. (such as P. aeruginosa), Acenitobacter spp. (such as A. baumannii), Salmonella spp. (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

31. A method of controlling at least one undesirable microbe by exposing the at least one undesirable microbe to:

(a) an effective amount of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof wherein the active variant comprises a bacterial strain having a genome within a Mash distance of about 0.015, wherein said effective amount comprises about 10³ CFU/gram to about 10¹² CFU/gram or about 10³ CFU/ml to about 10¹² CFU/ml;

(b) an effective amount of at least one of a spore, or a forespore, or a combination of cells, forespores and/or spores from at least one of AIP088262, AIP068104, AIP016597, AIP004816, AIP053802, AIP004634, AIP006035, AIP029002, AIP066414, AIP093093, AIP022568, AIP032005 or an active variant of any thereof, wherein the active variant comprises a bacterial strain having a genome within a Mash distance of about 0.015, and wherein said effective amount comprises about 10³ CFU/gram to about 10¹² CFU/gram or about 10³ CFU/ml to about 10¹² CFU/ml; and/or

(c) an effective amount of a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

wherein said effective amount controls the at least one undesirable microbe. 32. The method of embodiment 31, wherein the undesirable microbe comprises at least one pathogenic bacterium.

33. The method of embodiment 32, wherein the pathogenic bacteria comprises a strain selected from the group consisting of Clostridia spp. (such as C. perfringens and C. dificile), Burkeholderia spp. (such as B. pseudomallei), Pseudomonas spp. (such as P. aeruginosa), Acenitobacter spp. (such as A. baumannii), Salmonella spp. (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

34. A method of treating or preventing a disease caused by at least one undesirable microbe, comprising exposing the at least one undesirable microbe to:

(a) an effective amount of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof wherein the active variant comprises a bacterial strain having a genome within a Mash distance of about 0.015,

wherein said effective amount comprises about 10³ CFU/gram to about 10¹² CFU/gram or about 10³ CFU/ml to about 10¹² CFU/ml;

(b) an effective amount of at least one of a spore, or a forespore, or a combination of cells, forespores and/or spores from at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374 or an active variant of any thereof, wherein the active variant comprises a bacterial strain having a genome within a Mash distance of about 0.015, and wherein said effective amount comprises about 10³ CFU/gram to about 10¹² CFU/gram or about 10³ CFU/ml to about 10¹² CFU/ml; and/or

(c) an effective amount of a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

wherein said effective amount treats or prevents the disease caused by the undesirable microbes.

35. The method of embodiment 34, wherein the undesirable microbe comprises at least one pathogenic bacteria.

36. The method of embodiment 35, wherein the pathogenic bacteria is a strain selected from the group consisting of Clostridia spp. (such as C. perfringens and C. dificile), Salmonella spp (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

37. A method of controlling at least one undesirable microbe within an animal's gastrointestinal tract, the method comprising administering to the animal a composition comprising:

(a) an effective amount of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof wherein the active variant comprises a bacterial strain having a genome within a Mash distance of about 0.015, wherein said effective amount comprises about 10³ CFU/gram to about 10¹² CFU/gram or about 10³ CFU/ml to about 10¹² CFU/ml;

(b) an effective amount of at least one of a spore, or a forespore, or a combination of cells, forespores and/or spores from at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374 or an active variant of any thereof, wherein the active variant comprises a bacterial strain having a genome within a Mash distance of about 0.015, and wherein said effective amount comprises about 10³ CFU/gram to about 10¹² CFU/gram or about 10³ CFU/ml to about 10¹² CFU/ml; and/or

(c) an effective amount of a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

wherein said effective amount controls the at least one undesirable microbe within the animal's gastrointestinal tract.

38. A method of treating or preventing a disease in an animal caused by an undesirable microbe, comprising administering to an animal infected with said undesirable microbe or at risk of developing an infection of said undesirable microbe a composition comprising an effective amount of:

(a) at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015, wherein said effective amount comprises about 10³ CFU/gram to about 10¹² CFU/gram or about 10³ CFU/ml to about 10¹² CFU/ml; and/or (b) at least one of a spore or a forespore, or a combination of cells, forespores and/or spores from at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015; wherein the active variant comprises a bacterial strain having a genome within a Mash distance of about 0.015, wherein said effective amount comprises about 10³ CFU/gram to about 10¹² CFU/gram or about 10³ CFU/ml to about 10¹² CFU/ml; and/or (c) an effective amount of a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

wherein the effective amount treats or prevents the disease caused by the undesirable microbe. 39. The method of embodiment 37 or 38, wherein the animal is a vertebrate. 40. The method of embodiment 39, wherein the vertebrate is a mammal. 41. The method of embodiment 40, wherein the mammal is a horse, dog, cat, cow, goat, sheep, pig, deer, or human.

42. The method of embodiment 39, wherein the vertebrate is a bird or a reptile.

43. The method of embodiment 42, wherein the bird is a chicken, turkey, duck, goose, guinea fowl, ostrich, emu, quail, chukar, pheasant, grouse, Cornish hen, or partridge.

44. The method of embodiment 42, wherein the reptile is a lizard or snake.

45. The method of embodiment 39, wherein the animal is an aquatic animal.

46. The method of embodiment 45, wherein the aquatic animal is a salmon, trout, flounder, catfish, tilapia, ornamental fish, shrimp, crab, or lobster.

47. The method of any one of embodiments 38-46, wherein said treatment reduces at least one symptom of disease caused by the undesirable microbe.

48. The method of claim 47, wherein said symptom comprises, weight loss, reduction of growth rate, mortality, reduction of milk production, inflammation, loss of pregnancy, skin lesions, coughing, sneezing, diarrhea, increased body temperature or a combination thereof 49. A method of improving the health of an animal, comprising administering to the animal a composition comprising:

(a) an effective amount of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof wherein the active variant comprises a bacterial strain having a genome within a Mash distance of about 0.015, wherein said effective amount comprises about 10³ CFU/gram to about 10¹² CFU/gram or about 10³ CFU/ml to about 10¹² CFU/ml;

(b) an effective amount of at least one of a spore, or a forespore, or a combination of cells, forespores and/or spores from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374 or an active variant of any thereof, wherein the active variant comprises a bacterial strain having a genome within a Mash distance of about 0.015, and wherein said effective amount comprises about 10³ CFU/gram to about 10¹² CFU/gram or about 10³ CFU/ml to about 10¹² CFU/ml; and/or

(c) an effective amount of a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, wherein the active variant comprises a bacterial strain having a genome within a Mash distance of about 0.015;

wherein said effective amount improves the health of the animal.

50. The method of embodiment 49, wherein improvement of the health of the animal is determined by increased growth rate, increased average weight gain, higher feed intake, improved feed conversion ratio, higher feed conversion efficiency, and/or improved nutrient digestibility when compared to a control animal or group of animals that were not provided with an effective amount of said composition.

51. The method of embodiment 49 or 50, wherein the animal is a vertebrate.

52. The method of embodiment 51, wherein the vertebrate is a mammal. 53. The method of embodiment 52, wherein the mammal is a horse, dog, cat, cow, goat, sheep, pig, deer, or human.

54. The method of embodiment 51, wherein the vertebrate is a bird or a reptile. 55. The method of embodiment 53, wherein the bird is a chicken, turkey, duck, goose, guinea fowl, ostrich, emu, quail, chukar, pheasant, grouse, Cornish hen, or partridge.

56. The method of embodiment 55, wherein the reptile is a lizard or snake.

57. The method of embodiment 49 or 50, wherein the animal is an aquatic animal.

58. The method of embodiment 57, wherein the aquatic animal is a salmon, trout, flounder, catfish, tilapia, ornamental fish, shrimp, crab, or lobster.

The following examples are offered by way of illustration and not by way of limitation.

### EXAMPLES

### Example 1: Selection of Bacterial Strains

The bacterial strains of the invention were selected based on a number of criteria, including genomic profiling, antagonistic activity against S. enterica and also against a spectrum of virulent E. coli strains, acid and heat tolerance of the bacteria, acidification, lactic acid production, and ability to thrive and sporulate in selected media. To satisfy the majority of these criteria, bacterial strains had to be evaluated empirically, as described in the examples below. It could not be predicted, based either on taxonomy or genomic profiling of a given strain, how well it would perform in the empirical assays. Of over 1,500 bacterial strains initially evaluated empirically, 24 strains were determined to possess the desired combination of characteristics. These 24 strains are identified in Table 1 below and are characterized in the following examples.

TABLE 1
Bacterial strains of the invention

| Strain ID | NRRL No. | Date of Deposit | Taxonomic Designation |
|---|---|---|---|
| AIP088262 | B-67923 | Jan. 17, 2020 | Bacillus velezensis |
| AIP068104 | B-67922 | Jan. 17, 2020 | Bacillus subtilis |
| AIP016597 | B-67921 | Jan. 17, 2020 | Bacillus subtilis |
| AIP004816 | B-67920 | Jan. 17, 2020 | Bacillus subtilis |
| AIP053802 | B-67919 | Jan. 17, 2020 | Bacillus subtilis |
| AIP004634 | B-67918 | Jan. 17, 2020 | Bacillus subtilis |
| AIP006035 | B-67917 | Jan. 17, 2020 | Bacillus subtilis |
| AIP029002 | B-67916 | Jan. 17, 2020 | Bacillus subtilis |
| AIP066414 | B-67915 | Jan. 17, 2020 | Bacillus subtilis |
| AIP093093 | B-67914 | Jan. 17, 2020 | Bacillus subtilis |
| AIP022568 | B-67913 | Jan. 17, 2020 | Bacillus subtilis |
| AIP032005 | B-67912 | Jan. 17, 2020 | Bacillus subtilis |
| AIP012656 | B-67968 | Jun. 22, 2020 | Bacillus velezensis |
| AIP002364 | B-67967 | Jun. 22, 2020 | Bacillus velezensis |
| AIP044543 | B-67965 | Jun. 22, 2020 | Bacillus subtilis |
| AIP090377 | B-67964 | Jun. 22, 2020 | Bacillus subtilis |
| AIP048352 | B-67966 | Jun. 22, 2020 | Bacillus velezensis |
| AIP089343 | B-67962 | Jun. 22, 2020 | Bacillus pumilus |
| AIP007305 | B-67961 | Jun. 22, 2020 | Bacillus pumilus |
| AIP033189 | B-67958 | Jun. 22, 2020 | Bacillus amyloliquefaciens |
| AIP063641 | B-67963 | Jun. 22, 2020 | Bacillus subtilis |
| AIP087760 | B-67965 | Jun. 22, 2020 | Bacillus subtilis |
| AIP097873 | B-67960 | Jun. 22, 2020 | Bacillus inaquosorum |
| AIP056374 | B-67959 | Jun. 22, 2020 | Bacillus inaquosorum |

### Example 2: Bioinformatic Analysis of Bacterial Strains

Genomes of over 80,000 bacterial strains were profiled by bioinformatic analysis. Bioinformatic analysis based on the AMR Finder Plus tool available at NCBI identified genes associated with antimicrobial resistance (AMR). Any strains containing transmissible AMR genes, where the AMR gene was located on a plasmid or a transposon, were not elected for further consideration. Bioinformatic analysis also revealed the presence of desirable genomic features, such as biosynthetic gene clusters encoding for lichenysin, fengycin, or bacilysin. Other desirable genomic attributes included genes encoding for the biosynthetic production of short chain fatty acids, which are produced by the microbiota and contribute to gut health; genes encoding for carbohydrate-active enzymes; genes encoding for the ability to produce spores; and genes encoding for phytases, xylanases, lipases, amylases, and/or beta-glucanases, all of which may help with feed conversion.

### Example 3: Bacterial Strains have Antagonistic Activity Against Undesirable Microbes

Based on the results of the bioinformatic screen, about 1,500 bacterial strains were selected to empirically assay for antagonistic activity against undesirable microbes. Bioinformatic analysis is not predictive of the performance of any given strain in the antagonism assays. Antagonistic activity against pathogenic strains of Escherichia coli and Salmonella enterica was determined using a method based on the agar diffusion assay of Burkholder et al. (1966). Two antagonism assays were performed. In the first, bacterial strains of interested were assayed for antagonistic activity against E. coli strain MG1655 (ATCC Deposit No. 700926) and S. enterica strain LT2 (ATCC Deposit No. 19585). The first antagonism assay indicated if a bacterial strain of interest was antagonistic against multiple genera of pathogenic bacteria. The second antagonism assay was performed against E. coli strain MG1655 and other E. coli strains which are known to be pathogenic on pigs. This second assay indicated if a bacterial strain of interest was antagonistic against a diverse range of pathogenic E. coli strains.

Briefly, a pathogenic strain was embedded in 1% Mueller-Hinton agar medium in an ANSI footprint plate. The medium also contained 0.2% bromocresol purple to report a pH reduction. Bacterial strains were individually spotted at least 3 mm apart onto the medium and incubated at 39° C. for 48 hours. Each bacterial strain was scored for activity based on factors including colony size, colony morphology, lysis zone formation, inhibitory zone formation, and pH depression. Colony size is defined as the colony surface area of each strain growing on the agar medium containing the embedded pathogen strain. Lysis zone and inhibitory zone refer to zones of clearing that contain lysed pathogen cells or are entirely free of pathogen cells, respectively. The development of a yellow color around a test colony indicates a reduction in pH. Results for the bacterial strains of the invention are shown in Tables 2 and 3. Activity is indicated qualitatively as a “Yes” or “No” . The E. coli strain MG1655 and S. enterica strain LT2 are indicated. The pathogenic E. coli strains are indicated as “strain 1P”, “strain 2P”, etc.

TABLE 2
Antagonistic activity against pathogenic E. coli and S. enterica

| Strain ID | MG1655 | LT2 |
|---|---|---|
| AIP088262 | No | No |
| AIP068104 | Yes | Yes |
| AIP016597 | Yes | Yes |
| AIP004816 | Yes | Yes |
| AIP053802 | Yes | Yes |
| AIP004634 | Yes | Yes |
| AIP006035 | Yes | Yes |
| AIP029002 | Yes | Yes |
| AIP066414 | Yes | Yes |
| AIP093093 | No | Yes |
| AIP022568 | Yes | Yes |
| AIP032005 | Yes | Yes |
| AIP012656 | yes | yes |
| AIP002364 | yes | yes |
| AIP044543 | yes | yes |
| AIP090377 | yes | yes |
| AIP048352 | yes | yes |
| AIP089343 | yes | yes |
| AIP007305 | yes | no |
| AIP033189 | yes | yes |
| AIP063641 | yes | yes |
| AIP087760 | yes | yes |
| AIP097873 | yes | yes |
| AIP056374 | yes | yes |

TABLE 3
Antagonistic activity against pathogenic E. coli bacterial strains

| | Strain | Strain | Strain | Strain | Strain | Strain | Strain | Strain | Strain | Strain | |
|---|---|---|---|---|---|---|---|---|---|---|---|
| Strain ID | 1P | 2P | 3P | 4P | 5P | 6P | 7P | 9P | 9P | 10P | MG1655 |
| AIP088262 | No | Yes | Yes | Yes | Yes | Yes | Yes | Yes | Yes | Yes | No |
| AIP068104 | Yes | Yes | Yes | Yes | Yes | Yes | Yes | Yes | Yes | Yes | Yes |
| AIP016597 | No | Yes | Yes | Yes | Yes | Yes | Yes | Yes | Yes | Yes | Yes |
| AIP004816 | Yes | Yes | Yes | Yes | Yes | Yes | Yes | Yes | Yes | Yes | Yes |
| AIP053802 | Yes | Yes | Yes | Yes | Yes | Yes | Yes | Yes | Yes | Yes | Yes |
| AIP004634 | No | Yes | Yes | Yes | Yes | Yes | Yes | Yes | Yes | Yes | Yes |
| AIP006035 | No | Yes | Yes | Yes | Yes | Yes | Yes | Yes | Yes | Yes | Yes |
| AIP029002 | Yes | Yes | Yes | Yes | Yes | Yes | Yes | Yes | Yes | Yes | Yes |
| AIP066414 | No | Yes | Yes | Yes | Yes | Yes | Yes | Yes | Yes | Yes | Yes |
| AIP093093 | No | Yes | Yes | Yes | Yes | Yes | Yes | Yes | Yes | Yes | No |
| AIP022568 | No | Yes | Yes | Yes | Yes | Yes | Yes | Yes | Yes | Yes | Yes |
| AIP032005 | Yes | Yes | Yes | Yes | Yes | Yes | Yes | Yes | Yes | Yes | Yes |
| AIP012656 | Yes | Yes | Yes | Yes | Yes | Yes | Yes | Yes | Yes | Yes | Yes |
| AIP002364 | Yes | Yes | Yes | Yes | Yes | Yes | Yes | Yes | Yes | Yes | Yes |
| AIP044543 | Yes | Yes | Yes | Yes | Yes | Yes | Yes | Yes | Yes | Yes | Yes |
| AIP090377 | Yes | Yes | Yes | Yes | Yes | Yes | Yes | Yes | Yes | Yes | Yes |
| AIP048352 | Yes | No | No | Yes | No | Yes | No | Yes | Yes | Yes | Yes |
| AIP089343 | Yes | No | Yes | Yes | Yes | Yes | Yes | Yes | Yes | Yes | Yes |
| AIP007305 | Yes | Yes | Yes | No | Yes | Yes | Yes | Yes | Yes | Yes | Yes |
| AIP033189 | Yes | Yes | Yes | Yes | Yes | Yes | Yes | Yes | Yes | Yes | No |
| AIP063641 | Yes | Yes | Yes | Yes | Yes | Yes | Yes | Yes | Yes | Yes | No |
| AIP087760 | Yes | Yes | Yes | Yes | Yes | Yes | Yes | Yes | Yes | Yes | No |
| AIP097873 | Yes | Yes | Yes | Yes | Yes | Yes | Yes | Yes | Yes | Yes | Yes |
| AIP056374 | Yes | Yes | Yes | Yes | Yes | Yes | Yes | Yes | Yes | No | Yes |

### Example 4: Acid and Heat Tolerance Assay

Bacterial strains were inoculated into wells of a 96-well plate containing 800 μl unbuffered Mueller-Hinton Broth (uMHB) and grown at 30° C. with mild agitation for at least 48 hrs. Each strain was represented twice in a single 96-well plate. To induce sporulation, the 96-well plate was placed at 4° C. for at least 48 hours.

A sample of each sporulated strain culture was transferred to a fresh 96-well plate (the “heat plate”) and incubated at 80° C. for 20 minutes. After allowing the plate to cool, half the volume of each heat-treated sample was transferred to a 96-well plate (the “acid plate”) in the presence of the strong acid HCl. Following incubation at room temperature, the sample was neutralized with NaOH and the dilution to extinction method was used to identify cultures that remained viable and capable of robust growth at 39° C. for 48 hours. The scattered light (AU) reading was then measured to determine growth for each sample and compared to a negative control. The assay described above was repeated three times for each strain. All strains disclosed herein consistently exhibited vigorous or robust growth at 39° C. after 48 hours and were considered to have acid and heat tolerance.

### Example 5: Acidification and Lactic Acid Production

A BioLector® Pro (m2p-labs, Inc. Hauppauge, N.Y.) was used to determine acid production by a bacterial strain of interest as it grew over 24 hours. Bacterial strains were cultured in a selected media suitable for growing in large fermentation cultures and evaluated for 24 hours of growth in the BioLector®, during which time the pH of the media was measured following manufacturer's procotols. Strains were considered to have acidified the media if the pH of the media decreased during the 24 hours, especially if the pH reached the 5.0-6.0 range.

The lactic acid production of bacterial strains of interest was determined using a Yellow Springs Instrument (YSI) 2900 (YSI; Yellow Springs, Ohio) to measure lactate concentration (g/L) following manufacturer's protocols.

### Example 6. Methods of Culturing

A bacterial strain of interest was cultured in liquid media similar to that described in Example 5. The table below summarizes the incubation time, the concentration of bacteria achieved and percentage of sporulation. The concentration of bacteria was determined by measuring maximal (max) scattered light readings and correlating those values with optical density (O D₆₀₀). This was performed for two biological replicates and the average was determined. The percentage of sporulation was determined by averaging the sporulation percentage of two biological replicates.

TABLE 4
Sporulation of bacterial strains

| | | | Average |
|---|---|---|---|
| | Incubation | Average Max | Sporulation |
| Strain | time (hrs) | Optical Density | Percentage |
| AIP068104 | 72 | 33.67 | 100% |
| AIP016597 | 72 | 25.88 | 90% |
| AIP004816 | 72 | 30.35 | 80% |
| AIP053802 | 72 | 43.66 | 90% |
| AIP004634 | 72 | 31.39 | 80% |
| AIP006035 | 72 | 30.73 | 75% |
| AIP029002 | 72 | 50.58 | 80% |
| AIP088262 | 72 | 39.01 | 70% |
| AIP066414 | 72 | 31.74 | 80% |
| AIP093093 | 72 | 55.65 | 90% |
| AIP022568 | 72 | 43.03 | 80% |
| AIP032005 | 72 | 47.77 | 100% |
| AIP012656 | 72 | 38.65 | 90% |
| AIP002364 | 72 | 56.95 | 90% |
| AIP044543 | 72 | 34.03 | 90% |
| AIP090377 | 72 | 33.94 | 75% |
| AIP048352 | 72 | 45.32 | 82.5% |
| AIP089343 | 72 | 52.94 | 90% |
| AIP007305 | 72 | 45.26 | 90% |
| AIP033189 | 72 | 39.34 | 75% |
| AIP063641 | 72 | 41.06 | 90% |
| AIP087760 | 72 | 29.27 | 90% |
| AIP097873 | 72 | 30.26 | 82.5% |
| AIP056374 | 72 | 50.71 | 90% |

### Example 7: Bacillus Antagonism Assay

In some embodiments of the invention, the strains disclosed herein may be present in a composition as or used in a method as a combination, which comprises more than strain. Selected strains of the invention were evaluated to determine if growing them together affected their individual growth rate compared to growing them individually. To determine this, an antagonism assay similar to that described in Example 3 was performed. For these antagonism assays, a liquid culture of a disclosed strain was embedded into the assay medium, and selected strains were arrayed on the embedded agar. Zones of inhibition were scored on a 0 to 3 scale. The assay was performed three times and zones of inhibition were averaged, as shown in Table 5 below. The average antagonism score was calculated based on the zones of inhibition for all assayed strains and normalized to 1, where strain AIP033189, which had the highest zones of inhibition, has a value of 1 and strain AIP006035, which had zero for zones of inhibition, has a value of 0. Strains AIP004634, AIP087760, and AIP033189 have the highest average antagonism scores, suggesting that these three strains may impact the growth of other strains when cultured together. Strains AIP004816, AIP053802, and AIP006035 have the lowest average antagonism scores, indicating these three strains are likely to grow well in culture together and/or with many of the other strains disclosed herein.

TABLE 5
Bacillus antagonism assay

| | | Antagonistic Strain | | | | | |
|---|---|---|---|---|---|---|---|
| | | AIP053802 | AIP004816 | AIP022568 | AIP088262 | AIP044543 | AIP012656 |
| Embedded | AIP088262 | 0 | 0 | 0 | 0 | 0 | 0 |
| sStrain | AIP004816 | 0 | 0 | 0.67 | 0 | 0 | 0 |
| | AIP022568 | 0 | 0 | 0 | 0 | 0 | 0 |
| | AIP004634 | 0 | 0 | 0 | 0 | 0 | 0 |
| | AIP006035 | 0.67 | 0.67 | 0.67 | 0.67 | 0 | 0.33 |
| | AIP053802 | 0 | 0 | 0 | 0 | 0 | 0 |
| | AIP012656 | 0 | 0 | 0 | 0 | 0 | 0 |
| | AIP044543 | 0 | 0 | 0.67 | 0 | 0 | 0 |
| | AIP007305 | 0.67 | 0.33 | 0.67 | 0.33 | 0 | 0.33 |
| | AIP087760 | 0 | 0 | 0 | 0 | 0 | 0 |
| | AIP097873 | 0 | 0.67 | 0.33 | 0 | 0 | 0 |
| | AIP033189 | 0 | 0 | 0.67 | 0 | 0 | 0 |
| | Avg. | 0.11 | 0.13 | 0.29 | 0.08 | 0 | 0.05 |
| | Antagonism | | | | | | |
| | Score | | | | | | |

| | | Antagonistic Strain | | | | |
|---|---|---|---|---|---|---|
| | | AIP006035 | AIP004634 | AIP097873 | AIP087760 | AIP033189 |
| Embedded | AIP088262 | 0 | 1 | 0 | 0 | 0 |
| sStrain | AIP004816 | 0 | 0 | 0 | 1.67 | 2.67 |
| | AIP022568 | 0 | 1 | 0 | 0 | 2 |
| | AIP004634 | 0 | 0 | 1 | 1.33 | 2 |
| | AIP006035 | 0 | 1.33 | 1 | 1.33 | 2 |
| | AIP053802 | 0 | 1.33 | 0 | 1 | 0 |
| | AIP012656 | 0 | 1 | 0 | 0 | 2 |
| | AIP044543 | 0 | 0 | 0 | 1.33 | 0 |
| | AIP007305 | 0 | 1 | 1.33 | 2 | 0 |
| | AIP087760 | 0 | 1 | 0 | 0 | 0 |
| | AIP097873 | 0 | 1.33 | 0 | 1.67 | 2 |
| | AIP033189 | 0 | 1 | 0 | 1 | 0 |
| | Avg. | 0 | 0.79 | 0.26 | 0.89 | 1 |
| | Antagonism | | | | | |
| | Score | | | | | |

### Example 8: Combination Colony Antagonism Assay

Pairwise combinations of selected strains of the invention were assayed to determine combined antagonistic effect on pathogenic E. coli and S. enterica. This combination antagonism assay was similar to that described in Example 3, with the pathogenic E. coli or S. enterica embedded in the agar. For these antagonism assays, each of the two selected strains were cultured individually and mixed immediately before spotting on the surface of the agar. Each pairwise combination of strains, along with controls of each strain individually, was evaluated against three strains of E. coli described in Example 3 and the LT2 strain of S. enterica. Each combination colony and control single colony was evaluated by the size and clarity of the zone of inhibition. The size of each combination colony was also noted. The clearing size of the zone of inhibition for the combination colony was compared to that of the strain combined with itself, and calls were made determining if the combination colony performed better, the same, or worse than the individual strain, where the strain combined with itself was given a value of 1.00. Results are provided in Table 6 below. The following pairwise combinations showed larger clearing sizes compared to each strain alone: AIP004816 and AIP053802; AIP004816 and AIP097873; AIP004816 and AIP012656; AIP053802 and AIP087760; AIP006035 and AIP097873. The combination colonies that contained AIP088262 were unusual in that they tended to grow so large that a zone of inhibition was not visible.

TABLE 6
Combination colony antagonism assay

| | AIP088262 | AIP022568 | AIP004816 | AIP053802 | AIP004634 | AIP006035 |
|---|---|---|---|---|---|---|
| AIP088262 | 1.00 | 0.36 | 0.60 | 0.51 | 0.46 | 0.32 |
| AIP022568 | 0.36 | 1.00 | 0.76 | 0.70 | 0.92 | 0.70 |
| AIP004816 | 0.60 | 0.76 | 1.00 | 1.33 | 0.86 | 0.84 |
| AIP053802 | 0.51 | 0.70 | 1.33 | 1.00 | 0.73 | 0.85 |
| AIP004634 | 0.46 | 0.92 | 0.86 | 0.73 | 1.00 | 1.00 |
| AIP006035 | 0.32 | 0.70 | 0.84 | 0.85 | 1.00 | 1.00 |
| AIP012656 | 0.63 | 0.68 | 1.12 | 0.39 | 0.49 | 1.03 |
| AIP044543 | 0.28 | 0.55 | 0.65 | 0.77 | 0.71 | 0.82 |
| AIP033189 | 0.15 | 0.85 | 0.72 | 0.79 | 0.55 | 1.00 |
| AIP087760 | 0.24 | 1.00 | 0.83 | 1.08 | 0.87 | 0.71 |
| AIP097873 | 0.38 | 0.92 | 1.24 | 0.70 | 0.90 | 1.26 |
| | | AIP012656 | AIP044543 | AIP033189 | AIP087760 | AIP097873 |
| | AIP088262 | 0.63 | 0.28 | 0.15 | 0.24 | 0.38 |
| | AIP022568 | 0.68 | 0.55 | 0.85 | 1.00 | 0.92 |
| | AIP004816 | 1.12 | 0.65 | 0.72 | 0.83 | 1.24 |
| | AIP053802 | 0.39 | 0.77 | 0.79 | 1.08 | 0.70 |
| | AIP004634 | 0.49 | 0.71 | 0.55 | 0.87 | 0.90 |
| | AIP006035 | 1.03 | 0.82 | 1.00 | 0.71 | 1.26 |
| | AIP012656 | 1.00 | 0.31 | 0.73 | 0.60 | 0.72 |
| | AIP044543 | 0.31 | 1.00 | 0.62 | 0.44 | 0.48 |
| | AIP033189 | 0.73 | 0.62 | 1.00 | 1.08 | 0.78 |
| | AIP087760 | 0.60 | 0.44 | 1.08 | 1.00 | 0.96 |
| | AIP097873 | 0.72 | 0.48 | 0.78 | 0.96 | 1.00 |

### Example 9: Zinc, Copper, and Tylosin Compatibility

The relative compatibility of selected strains of the invention with common feed additives, namely Zinc (ZnSO₄), Copper (CuSO₄), Tylosin, and a mixture of all three, was determined. The minimal inhibitory concentration (MIC) of each compound was determined by growing each strain in a gradient concentration of the compound. A similar experiment was performed with a mixture of all three (ZnCuTy). This experiment was performed three times, independently, and the geometric mean of each MIC is shown in parts per million in Table 7 below. The ZnCuTy mixture is reported as the concentration of zinc. A higher value indicates a higher concentration of the compound was required to prevent growth of the assayed strain.

TABLE 7
MIC for zinc, copper, and Tylosin

| | Zn | Cu | Ty | ZnCuTy |
|---|---|---|---|---|
| Strain ID | (ppm) | (ppm) | (ppm) | (ppm Zn) |
| AIP088262 | 625 | 3175 | 17.3 | 248 |
| AIP004816 | 625 | 4000 | 34.6 | 312 |
| AIP053802 | 787 | 4000 | 34.6 | 496 |
| AIP004634 | 625 | 4000 | 27.5 | 312 |
| AIP006035 | 625 | 4000 | 21.8 | 312 |
| AIP022568 | 625 | 3175 | 27.5 | 393 |
| AIP012656 | 625 | 3175 | 17.3 | 312 |
| AIP044543 | 625 | 3175 | 17.3 | 312 |
| AIP033189 | 787 | 4000 | 34.6 | 393 |
| AIP087760 | 787 | 4000 | 43.7 | 393 |
| AIP097873 | 625 | 4000 | 17.3 | 312 |

### Example 10: Nursery Pig Feeding Trial

A pig feeding trial is conducted to assess the effects of a direct-fed microbial (DFM) feed additive comprising a bacterial strain described herein, namely AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a combination thereof, on body weight gain, feed intake, and feed efficiency of nursery pigs. See for example U.S. Pat. No. 10,357,046, incorporated by reference in its entirety herein. Mixed-sex pens of pigs (PIC 337×C29) with a starting weight of 13.9±2.3 lb are sorted by weight into replicates based on body weight to create 12 replicates of 23-26 mixed-sex pigs/pen (6.7 ft2/pig stocking density). Each pen has a 3-space dry box feeder and 2 swinging water nipples. Pens are then randomly allotted a dietary treatment and immediately started on the study. Pens remain on dietary treatments until the end of the experimental period. Diets are formulated to meet or exceed NRC (1988) requirements. For the diets comprising a DFM comprising AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or a combination thereof, the strain(s) is added to the diet at 7.3×10⁷ CFU/kg feed and supplied approximately 1×10⁸ CFU/head/day based on average daily feed intake (ADFI).

The trial is run from weaning to six weeks post-weaning. Total pen weights are recorded at allotment of the experiment and at regular intervals during the experimental period. Period feed intakes corresponded with pen weight periods. Pen weights, feed delivered, and feed-in-feeder for each pen are used to calculate ADG, ADFI, and F/G ratio. All morbidity and mortality is recorded, along with any major health issues.

Each experiment is statistically analyzed individually. Initial data analysis is performed for all metrics to determine normality of distribution and outliers (±>3 standard deviations in difference from the grand mean) using the Univariate procedure of SAS (SAS Institute, Inc., Cary, N.C.). Body weights and cumulative growth rates, feed intakes, and feed/gain ratios are analyzed according to randomized complete-block designs using the Mixed procedure of SAS, with the main effect of diet and random effect of replicate. Serial body weights, growth rates, feed intakes, and feed/gain ratios are analyzed similarly, with week or period included as a repeated measure. Morbidity, mortality, and other health-related metrics are analyzed as nonparametric data using the NParlway procedure of SAS.

### Example 11: Grower-Finisher Pig Feeding Trial

A pig feeding trial is conducted to assess the effects of a DFM feed additive comprising a bacterial strain described herein, namely AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a combination thereof, on body weight gain, feed intake, and feed efficiency of grower-finisher pigs. See for example U.S. Pat. No. 9,089,151, incorporated by reference in its entirety herein. Approximately 180 pigs (Monsanto Choice Genetics GPK 35 females mated to EB Ultra sires) are blocked into three weight blocks by initial body weight and penned in groups of 5 pigs/pen at the completion of the nursery period. Pigs are moved to a wean-to-finish facility and housed 5 pigs/pen in totally slatted pens (1.52 m×3.05 m) equipped a single-hole feeder, and wean-to-finish cup waterers. Initial minimum ambient room temperature is maintained at approximately 78° F. During the finishing phase, minimum temperature is further reduced to 70° F. Feed and water are available freely throughout the study.

One of two dietary treatments are assigned to each pen (18 pens/treatment) within each block, and administered during Phase 1 (50 to 90 lbs), Phase 2 (90 to 130 lbs), Phase 3 (130 to 180 lbs), Phase 4 (180 to 230 lbs) and Phase 5 (230 lbs to market at approximately 270 lbs). The two dietary treatments consist of a basal control diet devoid of a DFM comprising a bacterial strain described herein, and the basal diet with a DFM comprising a bacterial strain described herein, in a five phase grower-finisher pig study. Diets are formulated to meet or exceed NRC (1988) requirements and consisted predominately of corn, soybean meal, and DDGS at 47%, 18.6%, and 30% of the diet, respectively. For the diets comprising a DFM comprising AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a combination thereof, the strain(s) is added to the diet at 7.3×10⁷ CFU/kg feed and supplied approximately 1×10⁸ CFU/head/day based on average daily feed intake (ADFI). Data collected are average daily gain, average daily feed intake, and feed required per unit of gain during each of the five growing-finishing phases. Pigs are removed from the study when the average pig weight of the entire barn reaches approximately 270 lbs.

Performance data are analyzed as a randomized complete block design with pen as the experimental unit and blocks based on initial body weight. Analysis of variance is performed using the GLM procedures of SAS.

### Example 12: Broiler Chicken Feeding Trials

A boiler chicken feeding trial is conducted to assess the effects of DFM feed additive comprising a bacterial strain described herein, namely AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a combination thereof, on weight gain, caloric efficiency, and survivability of broiler chickens. See for example U.S. Publication 2017/0079308, incorporated by reference in its entirety herein. Approximately 2100 hatch rooster chickens (Cobb 500 genetics) were evaluated up to 7 weeks of age. For this investigation, a total of 60 pens (6×6 ft, 35 ft2/pen—subtracting 1 sq ft for feeder space) were utilized, with a group size of 35 birds per pen. The pens were equipped with a dry tube feeder (30-lb feed capacity) with a total feeder space of 50 in. (1.7 in./bird) and 5 nipple drinkers/pen (7 birds/nipple). Birds were assigned to pen based on day old chick weight. Initial pen weight of all replicate pens have a maximum of range of 30 grams. Pens were then randomly allotted to dietary treatment from within replicate and immediately started on the study. Pens remained on dietary treatments until the end of the experiment.

Dietary treatments consist of a basal control diet devoid of a DFM comprising a bacterial strain described herein, and the basal diet with a DFM comprising a bacterial strain described herein. Basal control diet comprises yellow-dent corn, soybean meal, corn low-oil DDGS, and porcine meat and bone meal (Producers Cooperative, Bryan, Tex.) and poultry fat (Griffin Industries). All diets were formulated to be adequate in SID Lys (%, NRC, 1994) and the other essential AA, available P, and Ca. For the diets comprising a DFM comprising AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a combination thereof, the strain(s) is added to the diet at 7.3 x10⁸ CFU/g feed. The DFM test materials were supplemented to the final diets at the expense of corn. Diet components were mixed in a horizontal mixer. Each diet was pelleted at 185° F. following 30 s of conditioning. The diets for weeks 1-2 were crumbled following pelleting. In the starter phase, the pellet stability of the DFM formulation was determined. The samples were obtained as follows and sent for analysis: A 2-lb sample of Mash was obtained at the start and 5 samples (1-lb per sample) of pelleted feed were obtained during the course of each pelleting run for each DFM-containing treatment. Samples of pelleted feed were collected following a brief acclimation in each new pelleting run and 1.0 lb from each 2.0 lb sample combined to form a pooled sample for each treatment. Pelleted feed samples were allowed to cool to room temperature before they were sealed for shipping. Both Mash & Pelleted feed samples were sent for analysis. Each feedstuff was sampled for nutrient and mycotoxin analyses at each point of manufacturing of experimental diets, and final experimental diets were sampled.

To determine the effects of the DFM feed additive comprising a bacterial strain described herein, total pen weights are determined at the start. Pen weight, feed disappearance, and caloric efficiency (kcal/lb gain) are determined at 2,4, 6, and 7 weeks post-hatching. The feed/gain ratio is adjusted for mortality by the following equation: Total feed consumed/(pen weight gain+mortality weight). Morbidity and mortality were also determined.

For carcass performance, A subset of each replicate-pen was processed for the determination of carcass, fat pad, and breast meat yield. Six broilers per replicate pen were randomly selected for yield determination. Growth and carcass data was analyzed using a randomized complete-block design, live weight (by pen) as the replication factor, and 10 replicates.

Statistical Procedures were carried out as follows: Prior to analysis, all data was checked for outliers. Any observation >3 standard deviations in difference from the grand mean for that metric was removed from the dataset. Cumulative body weight, growth, carcass, and economic performance were analyzed as a RCBD with six (6) treatments and 10 replicates. Morbidity, mortality, and other health-related metrics were analyzed as non-parametric data. Data were subjected to a one-way ANOVA and separated using Fisher's LSD.

### Example 13: Calf Feeding Trial

A calf feeding trial is conducted to assess the effects of a DFM feed additive comprising a bacterial strain described herein, namely AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a combination thereof, on body weight gain and presence of pathogenic microbes within the calves. See for example U.S. Pat. No. 8,540,981, incorporated by reference in its entirety herein. The trial is conducted in a 120 stall mechanically ventilated veal production facility, with 120 sale barn sourced Holstein bull calves randomly and equally placed in treatment and control groups. Individual scale weights are measured at the start of the feeding trial and all calves in even-numbered stalls receive 1 gram (1×10⁹ CFU) of the bacteria strains described herein, namely AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a combination thereof, in the morning milk feeding and each feeding thereafter for a total dose of 2×10⁹ CFU/animal/day. The every-other-calf study design eliminates variability in calf placement, ventilation or grower feeding practices. All calves start directly on milk replacer at placement (w/o the direct-fed microbial). The calves are started on 6.4 oz (3 lbs solution) per calf per feeding of a 20:20, all milk formula (Anderson Calf Milk 20:20 non-medicated milk replacer with BioMos 3.4 grams/day at 10 oz feeding rate). Feeding rate increases approximately 0.5 oz daily over a 10 day period to a maximum of 10 oz milk replacer per feeding (5 lbs solution). The 10 oz feeding rate is continued through 53 days when feeding rate is reduced to once a day. Calves are fully weaned at 55 days. Milk replacer is mixed with hot water and fed at approximately 5:30 AM and 5:00 PM each day. The grower is provided with individual, foil, heat-sealed pouches that contained 60 grams of the direct-fed microbial, which is a water-soluble formulation containing 1 billion CFU/g of carrier, which may comprise bakers sugar, dextrose and/or baylith. With the exception of addition of the DFM feed additive, milk solutions are identical for the group receiving the DFM and the control group throughout the trial.

Calves are individually ear tagged with corresponding stall number upon placement. Twenty pairs (40 calves) are selected for collection of fecal swabs. Swabs are collected on all 40 calves at day 6, 8 (peak scours) and 15 in the trial. Pairs of calves are selected to be evenly dispersed throughout the room; in each pair one is a treatment and a control calf. Swabs are screened and the presence of pathogenic organisms Clostridium perfringens type A, B, C & D, Salmonella spp., and/or virulent strains of E. coli is determined using multiplex PCR analysis. Blood samples from the same 40 calves are drawn at day 6 of the trial using serum separator tubes. Samples are analyzed for total protein by a recognized industry expert regarding calf immunology. The weights of all calves are determined at the start of the study, at day 21, and at day 52. All treatments, feed refusals, and death losses are recorded.

### Example 14: Dairy Cow Feeding Trial

A dairy cow feeding trial is conducted to assess the effects of a DFM feed additive comprising a bacterial strain described herein, namely AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a combination thereof, on cow health, milk production, and reproduction parameters on dairy cows. See for example U.S. Publication 2015/0216916, incorporated by reference in its entirety herein. The trial is conducted on a herd of dairy cows ranging in size from 600 to 800 cows.

Milk production, reproduction, and health metrics are monitored on the entire herd via Dairy Comp 305 (Valley Agricultural Software, Tulare, Calif.) with a period of 3 months pre-DFM feed additive serving as the baseline. The entire herd is then monitored for milk production, milk components, health, and reproductive performance for 10 months during which cows received daily DFM feed additive, measuring weekly individual cow milk production, monthly DHIA milk components, and monthly health events. During the Bacillus supplementation period, cows received 15 grams of the Bacillus product daily to provide 7.35×10⁹ CFU per head per day of live AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, or an active variant of any thereof, or a combination thereof, within a standard corn silage and alfalfa hay-based total mixed ration. Reproductive metrics are evaluated during a 6-month period over consecutive years, during which pre-DFM feed additive is compared to post-DFM feed additive supplementation to control for seasonal variation.

Production and component data are analyzed by the MIXED procedure of SAS looking for fixed effects of treatment and the interaction of treatment*month*year, using days in milk (DIM) as covariate. Health and reproductive metrics are evaluated by the GLIMMIX procedure of SAS. Significance between means is defined at p≤0.05 and trends are indicated when p≤0.1.

### Example 15: In Vivo Evaluation of Bacillus Strains

Three proof of principle (POP) animal trials, POP 1, POP2 and POP3 were conducted for in vivo evaluation of Bacillus strains described herein. Fifteen Bacillus strains that were evaluated in the POP 1, POP2 and POP3 studies were AIP053802, AIP006035, AIP088262, AIP004816, AIP004634, AIP022568, AIP087760, AIP093093, AIP012656, AIP032005, AIP066414, AIP033189, AIP016597, AIP097873, and AIP044543. Each experiment consisted of seven treatments. These treatments included five Bacillus strains tested individually, an untreated negative control, and a positive control that received conventional antimicrobial feed additives (zinc oxide, copper sulphate, and tylosin). For the POP 1 study, each treatment had eight pens with five pigs per pen. For POP2 and POP3 studies, each treatment had seven pens with five pigs per pen.

Weaned piglets, approximately 21 days of age, were sourced from a local herd with a history of E. coli-associated post-weaning diarrhea. Pigs were randomly assigned to treatment groups, while balancing for sex within each treatment. Pigs were fed a mash diet in three dietary phases; Phase 1 (study days 0 to 7), Phase 2 (study days 7 to 21), and Phase 3 (study days 21 to 42).

The test article was prepared by growing each strain in a shake flask, inducing sporulation, concentrating, and spraying onto a ground rice hull carrier. The spores were mixed into the feed to a final concentration of 10⁵ colony forming units (CFU) per gram of feed.

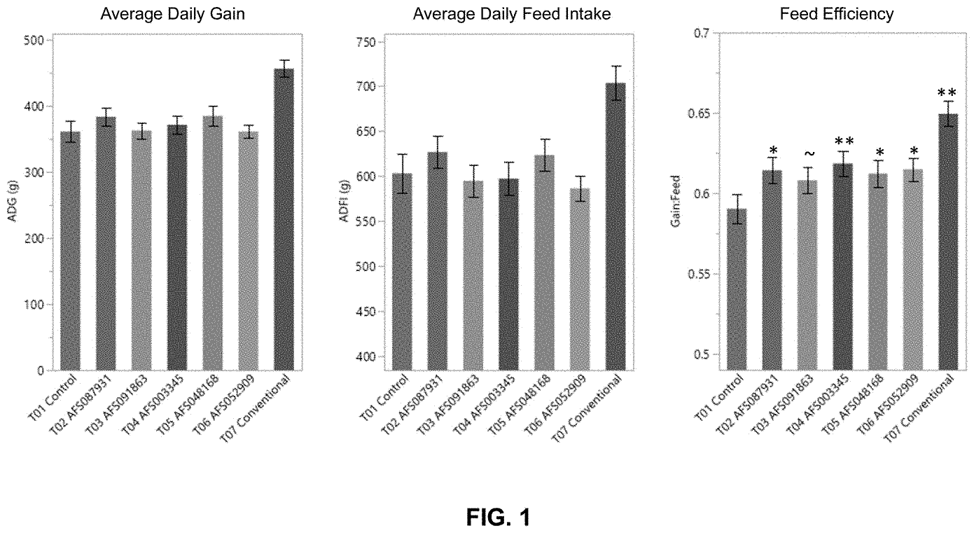

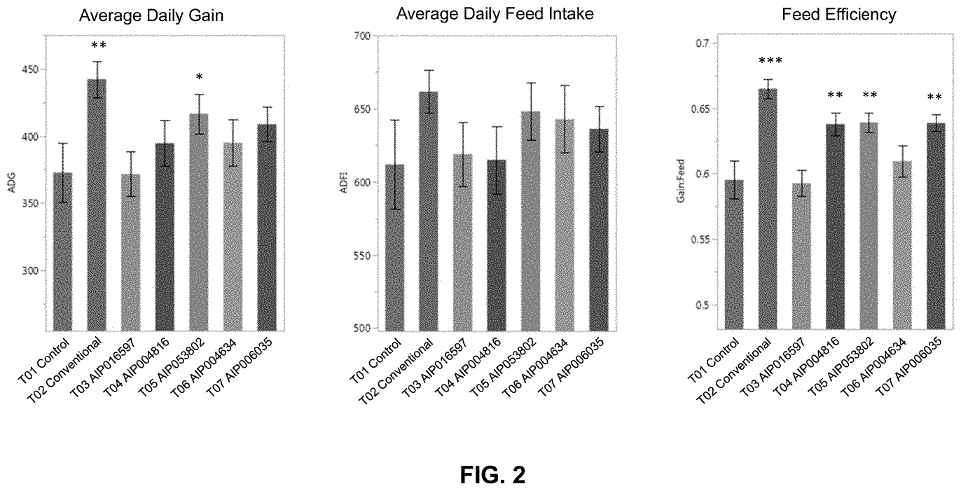

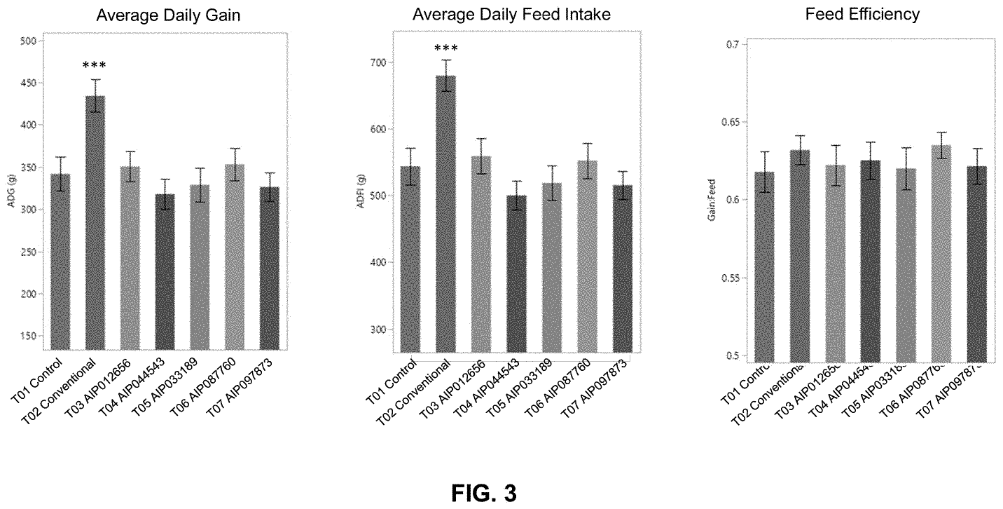

Growth performance was measured for individual pigs and by pen. The key performance metrics were average daily gain (ADG), average daily feed intake (ADFI), and feed efficiency measured by the ratio of gain to feed (G:F). Individual ADFI was calculated according to the procedure from Lee et al. (Asian-Australas J Anim Sci. 2016; 29(12): 1756-1760). Growth performance, as measured in POP1, POP2, and POP3 studies, is provided in FIGS. 1, 2, and 3, respectively. A summary of the growth performance is provided in Table 8 below.

TABLE 8
In vivo Growth Performance Summary

| | | ADG (g) | | | ADFI (g) | | | Gain:Feed | | |
|---|---|---|---|---|---|---|---|---|---|---|
| | | | | P-value | | | P-value | | | P-value |
| | | | % Change | vs | | % Change | vs | | % Change | vs |
| Study | Treatment | Mean | vs Control | Control | Mean | vs Control | Control | Mean | vs Control | Control |
| POP1 | Control | 361 | na | na | 603 | na | na | 0.590 | na | na |
| | AIP088262 | 383 | 6.1% | 0.2541 | 627 | 3.9% | 0.3583 | 0.614 | 4.0% | 0.035 |
| | AIP066414 | 362 | 0.3% | 0.9479 | 595 | −1.4% | 0.735 | 0.608 | 3.0% | 0.0943 |
| | AIP093093 | 371 | 2.8% | 0.5834 | 597 | −1.0% | 0.8093 | 0.619 | 4.8% | 0.0111 |
| | AIP022568 | 385 | 6.5% | 0.2226 | 624 | 3.4% | 0.425 | 0.612 | 3.7% | 0.0528 |
| | AIP032005 | 361 | −0.1% | 0.9921 | 586 | −2.8% | 0.5011 | 0.615 | 4.2% | 0.0219 |
| POP2 | Control | 380 | na | na | 625 | na | na | 0.595 | na | na |
| | AIP016597 | 371 | −2.4% | 0.9642 | 618 | −1.1% | 0.8154 | 0.593 | −0.4% | 0.8612 |
| | AIP004816 | 395 | 4.0% | 0.3391 | 616 | −1.3% | 0.9174 | 0.638 | 7.2% | 0.0032 |
| | AIP053802 | 420 | 10.5% | 0.0571 | 654 | 4.7% | 0.2307 | 0.639 | 7.4% | 0.0024 |
| | AIP004634 | 395 | 4.1% | 0.3322 | 644 | 3.1% | 0.3046 | 0.610 | 2.5% | 0.3176 |
| | AIP006035 | 406 | 6.9% | 0.1178 | 632 | 1.1% | 0.4203 | 0.639 | 7.4% | 0.0027 |
| POP3 | Control | 342 | na | na | 544 | na | na | 0.618 | na | na |
| | AIP012656 | 351 | 2.6% | 0.7398 | 559 | 2.8% | 0.6647 | 0.622 | 0.7% | 0.7919 |
| | AIP044543 | 318 | −7.0% | 0.3691 | 500 | −8.0% | 0.2148 | 0.625 | 1.2% | 0.6586 |
| | AIP033189 | 329 | −3.8% | 0.6264 | 519 | −4.6% | 0.4759 | 0.620 | 0.4% | 0.8943 |
| | AIP087760 | 353 | 3.4% | 0.6684 | 552 | 1.6% | 0.8081 | 0.635 | 2.8% | 0.3084 |
| | AIP097873 | 326 | −4.6% | 0.5555 | 515 | −5.2% | 0.4151 | 0.621 | 0.6% | 0.8246 |

The level of post-weaning diarrhea challenge was assessed by fecal E. coli CFU counts. The effect of treatment on performance variables was assessed using a two-tail t-test between each treatment and the negative control group. A Wilcoxon rank-sum test was used to compare E. coli CFU counts between each treatment and the negative control. P-values less than 0.1 were considered statistically significant. Next, the efficacy of the strains was ranked based on the growth performance and E. coli CFU counts. A summary of strain efficacy, as determined from POP1, POP2, and POP3 studies, is provided in Table 9 below.

TABLE 9
Strain Efficacy Summary

| | | ADG vs Ctrl | G:F vs Ctrl | Change in E. coli |
|---|---|---|---|---|
| Study | Treatment | (%) | (%) | (log10) |
| 2 | AIP053802 | 10.5% | 7.4% | −0.8 |
| 2 | AIP006035 | 6.9% | 7.4% | −0.96 |
| 1 | AIP088262 | 6.1% | 4.0% | −0.27 |
| 2 | AIP004816 | 4.0% | 7.2% | −0.36 |
| 2 | AIP004634 | 4.1% | 2.5% | −1.12 |
| 1 | AIP022568 | 6.5% | 3.7% | 0.69 |
| 3 | AIP087760 | 3.4% | 2.8% | −0.14 |
| 1 | AIP093093 | 2.8% | 4.8% | −0.25 |
| 3 | AIP012656 | 2.6% | 0.7% | −0.60 |
| 1 | AIP032005 | −0.1% | 4.2% | −0.56 |
| 1 | AIP066414 | 0.3% | 3.0% | −0.09 |
| 3 | AIP033189 | −3.8% | 0.4% | −1.16 |
| 2 | AIP016597 | −2.4% | −0.4% | −0.82 |
| 3 | AIP097873 | −4.6% | 0.6% | −0.18 |
| 3 | AIP044543 | −7.0% | 1.2% | −0.01 |

What is claimed is:

1. A composition comprising:
(a) at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, and wherein said bacterial strain is present at about 10³ CFU/gram to about 10¹² CFU/gram or at about 10³ CFU/ml to about 10¹² CFU/ml;
(b) at least one of a spore, or a forespore, or a combination of cells, forespores, and/or spores from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374, and wherein said spore, forespore, or a combination of cells, forespores, and/or spores is present at about 10³ CFU/gram to about 10¹² CFU/gram or about 10³ CFU/ml to about 10¹² CFU/ml; and/or
(c) a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, AIP056374;
wherein an effective amount of said composition controls the growth of at least one undesirable microbe.

2. The composition of claim 1, wherein said composition comprises at least one of a cell, a spore, or a forespore, or a combination of cells, forespores, and/or spores of AIP004816 and/or AIP053802, and/or AIP006035.

3. The composition of claim 2, wherein said composition further comprises at least one of a cell, a spore, or a forespore, or a combination of cells, forespores, and/or spores of AIP088262 and/or AIP097873.

4. The composition of claim 1, wherein said composition comprises at least one of a cell, a spore, or a forespore, or a combination of cells, forespores, and/or spores of AIP004816 and AIP012656.

5. The composition of claim 1, wherein said composition comprises at least one of a cell, a spore, or a forespore, or a combination of cells, forespores, and/or spores of AIP088262, and/or AIP004816, and/or AIP053802.

6. The composition of claim 5, wherein said composition further comprises at least one of a cell, a spore, or a forespore, or a combination of cells, forespores, and/or spores of AIP006035 and/or AIP022568.

7. The composition of claim 1, wherein said composition comprises at least one of a cell, a spore, or a forespore, or a combination of cells, forespores, and/or spores of AIP088262, and/or AIP053802, and/or AIP006035.

8. The composition of claim 7, wherein said composition further comprises at least one of a cell, a spore, or a forespore, or a combination of cells, forespores, and/or spores of AIP087760.

9. The composition of claim 1, wherein said composition comprises a combination of at least one of a cell, a spore, or a forespore, or a combination of cells, forespores, and/or spores of at least two bacterial strains selected from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374.

10. The composition of claim 1, wherein said composition comprises a combination of at least one of a cell, a spore, or a forespore, or a combination of cells, forespores, and/or spores of at least three bacterial strains selected from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374.

11. The composition of claim 1, wherein said composition comprises a combination of at least one of a cell, a spore, or a forespore, or a combination of cells, forespores, and/or spores of at least four bacterial strains selected from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374.

12. The composition of claim 1, wherein said composition comprises a combination of at least one of a cell, a spore, or a forespore, or a combination of cells, forespores, and/or spores of at least five bacterial strains selected from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, and AIP056374.

13. The composition of any one of claims 1 to 12, wherein said bacterial strain, spore, forespore, or combination of cells, forespores, and/or spores, is present at about 10³ CFU/gram to about 10¹⁰ CFU/gram or at about 10³ CFU/ml to about 10¹⁰ CFU/ml.

14. The composition of any one of claims 1 to 13, wherein said bacterial strain, spore, forespore, or combination of cells, forespores, and/or spores, is present at about 10⁴ CFU/gram to about 10⁸ CFU/gram or at about 10⁴ CFU/ml to about 10⁸ CFU/ml.

15. The composition of any of claims 1 to 14, wherein said composition comprises a cell paste, a wettable powder, a spray dried formulation, a wettable granule formulation, a pelleted formulation, or a stable formulation.

16. The composition of any of claims 1 to 15, wherein said composition further comprises at least one or more of a carrier, proteins, carbohydrates, fats, other probiotics, prebiotics, enzymes, vitamins, immune modulators, milk replacers, minerals, amino acids, coccidiostats, acid-based products, and medicines.

17. The composition of any of claims 1 to 16, wherein said composition comprises at least one additional bacterial strain.

18. The composition of claim 17, wherein the additional bacterial strain is selected from the group consisting of a Bacillus spp, Enterococcus spp., Bifidobacterium spp., Lactobacillus spp., Streptococcus spp., Propionibacterium spp., Megasphaera spp., Prevotella spp., and a Pediococcus spp.

19. The composition of claim 17, wherein said additional bacteria strain is selected from the group consisting of Bacillus subtilis, Bacillus licheniformis, Bacillus lentus, Bacillus pumilus, Bacillus laterosporus, Bacillus coagulans, Bacillus alevi, Bacillus cereus, Bacillus clausii, Bacillus coagulans, Bacillus mojavensis, Bacillus velezensis Bacillus vallismortis, Bacillus amyloliquefaciens, Bacillus atropheus, Bacillus altitudinis, Bacillus inaquosorum, Bacillus safensis, Bacillus alcalophilus, Bacillus badius, Bacillus thurigiensis, Bifidobacterium adolescentis, Bifidobacterium animalis, Bifidobacterium bifidum, Bifidobacterium infantis, Bifidobacterium longum, Bifidobacterium pseudolongum, Bifidobacterium thermophilum, Lactobacillus alactosus, Lactobacillus alimentarius, Lactobacillus amylovorans, Lactobacillus amylophilus, Lactobacillus amylovorans, Lactobacillus acidophilus, Lactobacillus agilis, Lactobacillus animalis, Lactobacillus batatas, Lactobacillusbavaricus, Lactobacillus bifermentans, Lactobacillus bidifus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus curvatus, Lactobacillus coprohilus, Lactobacillus delbrueckii, Lactobacillus fermentum, Lactobacillus gasseri, Lactobacillus jugurti, Lactobacillus kefir, Lactobacillus lactis, Lactobacillus leichmannii, Lactobacillus mali, Lactobacillus malefermentans, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus tolerans, Lactobacillus torquens, Lactobacillus ruminis, Lactobacillus sake, Lactobacillus saliverius, Lactobacillus sharpeae, Lactobacillus sobrius, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Streptococcus cremoris, Streptococcus discetylactis, Streptococcus faecium, Streptococcus lactis, Streptococcus thermophilus, Streptococcus intermedius, Enterococcus faecium, Propionibacterium freudenreichii, Propionibacterium acidipropionici, Propionibacterium jensenii, Propionibacterium thoenii, Propionibacterium australiense, Propionibacterium avidum, Megasphaera elsdenil, Prevotella bryantii, and Pediococcus acidilactici.

20. The composition of claim 17, wherein said composition further comprises at least one of bacterial strain Bacillus subtilis PB6, B. subtilis C-3102, B. subtilis DSM 17299, Bacillus licheniformis DSM 17236, B. licheniformis DSMZ 5749, B. subtilis DSMZ 5750, B. subtilis DSM 29784, or an active variant of any thereof.

21. The composition of any of claims 1-20, wherein said undesirable microbe comprises at least one pathogenic bacteria.

22. The composition of claim 21, wherein said pathogenic bacteria comprises a strain selected from the group consisting of Clostridia spp. (such as C. perfringens and C. dificile), Burkeholderia spp. (such as B. pseudomallei), Pseudomonas spp. (such as P. aeruginosa), Acenitobacter spp. (such as A. baumannii), Salmonella spp (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

23. A composition comprising a spray dried formulation, comprising:
at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374; and/or,
at least one of a spore, or a forespore, or a combination of cells, forespores and/or spores from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374;
a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374;
wherein an effective amount of said composition controls the growth of at least one undesirable microbe.

24. A composition comprising a wettable power, comprising:
(a) at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, and wherein said bacterial strain is present at about 10³ CFU/gram to about 10¹² CFU/gram or at about 10³ CFU/ml to about 10¹² CFU/ml;
(b) at least one of a spore, or a forespore, or a combination of cells, forespores, and/or spores from any of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, and wherein said spore, forespore, or a combination of cells, forespores, and/or spores is present at about 10³ CFU/gram to about 10¹² CFU/gram or at about 10³ CFU/ml to about 10¹² CFU/ml; and/or
(c) a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374;
wherein an effective amount of said composition controls the growth of at least one undesirable microbe.

25. An isolated biologically pure culture of a bacterial strain comprising:
(a) AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, and wherein said bacterial strain is present at about 10³ CFU/gram to about 10¹² CFU/gram or at about 10³ CFU/ml to about 10¹² CFU/ml; or,
(b) a spore, or a forespore, or a combination of cells, forespores and/or spores from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or A1P056374, and wherein said spore, forespore, or a combination of cells, forespores, and/or spores is present at about 10³ CFU/gram to about 10¹² CFU/gram or at about 10³ CFU/ml to about 10¹² CFU/ml;
wherein an effective amount of said culture controls the growth of at least one undesirable microbe.

26. The isolated biologically pure culture of claim 25, wherein said undesirable microbe comprises at least one pathogenic bacteria.

27. The isolated biologically pure culture of claim 26, wherein said pathogenic bacteria is a strain selected from the group consisting of Clostridia spp. (such as C. perfringens and C. dificile), Burkeholderia spp. (such as B. pseudomallei), Pseudomonas spp. (such as P. aeruginosa), Acenitobacter spp. (such as A. baumannii), Salmonella spp (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

28. A bacterial culture grown from:
(a) AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374; or,
(b) a spore, or a forespore, or a combination of cells, forespores and/or spores from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374;
(c) wherein said bacterial culture controls the growth of at least one undesirable microbe.

29. The bacterial culture of claim 28, wherein said undesirable microbe comprises at least one pathogenic bacteria.

30. The bacterial culture of claim 29, wherein said pathogenic bacteria comprises a strain selected from the group consisting of Clostridia spp. (such as C. perfringens and C. dificile), Burkeholderia spp. (such as B. pseudomallei), Pseudomonas spp. (such as P. aeruginosa), Acenitobacter spp. (such as A. baumannii), Salmonella spp (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

31. A method of controlling at least one undesirable microbe by exposing the at least one undesirable microbe to:
(a) an effective amount of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, wherein said effective amount comprises about 10³ CFU/gram to about 10¹² CFU/gram or about 10³ CFU/ml to about 10¹² CFU/ml;
(b) an effective amount of at least one of a spore, or a forespore, or a combination of cells, forespores and/or spores from at least one of AIP088262, AIP068104, AIP016597, AIP004816, AIP053802, AIP004634, AIP006035, AIP029002, AIP066414, AIP093093, AIP022568, or AIP032005, and wherein said effective amount comprises about 10³ CFU/gram to about 10¹² CFU/gram or about 10³ CFU/ml to about 10¹² CFU/ml; and/or
(c) an effective amount of a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374;
wherein said effective amount controls the at least one undesirable microbe.

32. The method of claim 31, wherein the undesirable microbe comprises at least one pathogenic bacterium.

33. The method of claim 32, wherein the pathogenic bacteria comprises a strain selected from the group consisting of Clostridia spp. (such as C. perfringens and C. dificile), Burkeholderia spp. (such as B. pseudomallei), Pseudomonas spp. (such as P. aeruginosa), Acenitobacter spp. (such as A. baumannii), Salmonella spp (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

34. A method of treating or preventing a disease caused by at least one undesirable microbe, comprising exposing the at least one undesirable microbe to:
(a) an effective amount of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, wherein said effective amount comprises about 10³ CFU/gram to about 10¹² CFU/gram or about 10³ CFU/ml to about 10¹² CFU/ml;
(b) an effective amount of at least one of a spore, or a forespore, or a combination of cells, forespores and/or spores from at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, and wherein said effective amount comprises about 10³ CFU/gram to about 10¹² CFU/gram or about 10³ CFU/ml to about 10¹² CFU/ml; and/or
(c) an effective amount of a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374;
wherein said effective amount treats or prevents the disease caused by the undesirable microbes.

35. The method of claim 34, wherein the undesirable microbe comprises at least one pathogenic bacteria.

36. The method of claim 35, wherein the pathogenic bacteria is a strain selected from the group consisting of Clostridia spp. (such as C. perfringens and C. dificile), Burkeholderia spp. (such as B. pseudomallei), Pseudomonas spp. (such as P. aeruginosa), Acenitobacter spp. (such as A. baumannii), Salmonella spp (such as S. enterica, S. arizonae, S. typhirium, S. enteritidis, and S. bonglori), Listeria spp. (such as L. moncytogenes, L. seeligeri, and L. welshimeri), Escherichia spp, (such as E. coli) Enterococci spp. (such as E. faecalis and E. bonglori), Staphylococci spp. (such as S. aureus), Aeromonas spp, Streptococci spp, Campylobacter spp, Haemophilus spp, Brachyspira spp, and Vibrio spp.

37. A method of controlling at least one undesirable microbe within an animal's gastrointestinal tract, the method comprising administering to the animal a composition comprising:
(a) an effective amount of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, wherein said effective amount comprises about 10³ CFU/gram to about 10¹² CFU/gram or about 10³ CFU/ml to about 10¹² CFU/ml;
(b) an effective amount of at least one of a spore, or a forespore, or a combination of cells, forespores and/or spores from at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, and wherein said effective amount comprises about 10³ CFU/gram to about 10¹² CFU/gram or about 10³ CFU/ml to about 10¹² CFU/ml; and/or (c) an effective amount of a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374;
wherein said effective amount controls the at least one undesirable microbe within the animal's gastrointestinal tract.

38. A method of treating or preventing a disease in an animal caused by an undesirable microbe, comprising administering to an animal infected with said undesirable microbe or at risk of developing an infection of said undesirable microbe a composition comprising an effective amount of:
(a) at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, wherein said effective amount comprises about 10³ CFU/gram to about 10¹² CFU/gram or about 10³ CFU/ml to about 10¹² CFU/ml; and/or (b) at least one of a spore or a forespore, or a combination of cells, forespores and/or spores from at least one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, wherein said effective amount comprises about 10³ CFU/gram to about 10¹² CFU/gram or about 10³ CFU/ml to about 10¹² CFU/ml; and/or
(c) a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374;
wherein the effective amount treats or prevents the disease caused by the undesirable microbe.

39. The method of claim 37 or 38, wherein the animal is a vertebrate.

40. The method of claim 39, wherein the vertebrate is a mammal.

41. The method of claim 40, wherein the mammal is a horse, dog, cat, cow, goat, sheep, pig, deer, or human.

42. The method of claim 39, wherein the vertebrate is a bird or a reptile.

43. The method of claim 42, wherein the bird is a chicken, turkey, duck, goose, guinea fowl, ostrich, emu, quail, chukar, pheasant, grouse, Cornish hen, or partridge.

44. The method of claim 42, wherein the reptile is a lizard or snake.

45. The method of claim 39, wherein the animal is an aquatic animal.

46. The method of claim 45, wherein the aquatic animal is a salmon, trout, flounder, catfish, tilapia, ornamental fish, shrimp, crab, or lobster.

47. The method of any one of claims 38-46, wherein said treatment reduces at least one symptom of disease caused by the undesirable microbe.

48. The method of claim 47, wherein said symptom comprises, weight loss, reduction of growth rate, mortality, reduction of milk production, inflammation, loss of pregnancy, skin lesions, coughing, sneezing, diarrhea, increased body temperature or a combination thereof.

49. A method of improving the health of an animal, comprising administering to the animal a composition comprising:
(a) an effective amount of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, wherein said effective amount comprises about 10³ CFU/gram to about 10¹² CFU/gram or about 10³ CFU/ml to about 10¹² CFU/ml;
(b) an effective amount of at least one of a spore, or a forespore, or a combination of cells, forespores and/or spores from any one of AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374, and wherein said effective amount comprises about 10³ CFU/gram to about 10¹² CFU/gram or about 10³ CFU/ml to about 10¹² CFU/ml; and/or
(b) an effective amount of a supernatant, fermentation product, filtrate, or extract derived from a whole cell culture of at least one of bacterial strain AIP088262, AIP068104, AIP016597, AIP004816, AIP053802, AIP004634, AIP006035, AIP029002, AIP066414, AIP093093, AIP022568, AIP032005, AIP012656, AIP002364, AIP044543, AIP090377, AIP048352, AIP089343, AIP007305, AIP033189, AIP063641, AIP087760, AIP097873, or AIP056374;
wherein said effective amount improves the health of the animal.

50. The method of claim 49, wherein improvement of the health of the animal is determined by increased growth rate, increased average weight gain, higher feed intake, improved feed conversion ratio, higher feed conversion efficiency, and/or improved nutrient digestibility when compared to a control animal or group of animals that were not provided with an effective amount of said composition.

51. The method of claim 49 or 50, wherein the animal is a vertebrate.

52. The method of claim 51, wherein the vertebrate is a mammal.

53. The method of claim 52, wherein the mammal is a horse, dog, cat, cow, goat, sheep, pig, deer, or human.

54. The method of claim 51, wherein the vertebrate is a bird or a reptile.

55. The method of claim 53, wherein the bird is a chicken, turkey, duck, goose, guinea fowl, ostrich, emu, quail, chukar, pheasant, grouse, Cornish hen, or partridge.

56. The method of claim 55, wherein the reptile is a lizard or snake.

57. The method of claim 49 or 50, wherein the animal is an aquatic animal.

58. The method of claim 57, wherein the aquatic animal is a salmon, trout, flounder, catfish, tilapia, ornamental fish, shrimp, crab, or lobster.
